(19) United States
(12) Patent Application Publication
(10) Pub. No.: US 2007/0227537 A1
(43) Pub. Date: Oct. 4, 2007

(54) Systems and Methods for Facilitating Management of Respiratory Care

(75) Inventors: Stephen John Bemister, Kinburn (CA); Razmik Malek-Adamian, Nepean (CA); Thomas John Anderson, Dunrobin (CA); Ramin Sadafi, Ottawa (CA); Peter John Kelly, Nepean (CA); Marcel Ion Farcas, Kanata (CA); Zhiwei Peng, Ottawa (CA); Dan Chen, Nepean (CA); Faranak Khadjhepour, Kanata (CA)

Correspondence Address: BAKER BOTTS L.L.P.;PATENT DEPARTMENT, 98 SAN JACINTO BLVD., SUITE 1500, AUSTIN, TX, 78701-4039, US

(73) Assignee: Nellcor Puritan Bennett Incorporated

(21) Appl. No.: 11/566,218

(22) Filed: Dec. 2, 2006

Related U.S. Application Data
(60) Provisional application No. 60/741,641, filed on Dec. 2, 2005.

(51) Int. Cl. A61M 16/00; G05B 15/00

(52) U.S. Cl. 128/200.24; 700/84

(57) ABSTRACT

A method for configuring a protocol for use in providing a medical treatment to a patient is provided. A protocol may be selected for configuration, the protocol being associated with a medical treatment having a process flow including multiple steps. A protocol template may be created for the protocol, including selecting and arranging flowchart components to create a graphical flowchart representation of the process flow, and defining one or more variables for one or more steps in the process flow. The one or more variables may be linked to one or more existing objects such that when the protocol is executed, the existing objects are automatically accessed to facilitate execution of the protocol.

### TECHNICAL FIELD

The present disclosure is related generally to respiratory care, e.g., systems and methods for assisting the implementation and/or management of respiratory care using treatment protocols.

### BACKGROUND

Respiratory Therapists often use protocols to help manage the care they provide to their patients. Such protocols are typically based upon evidence-based medicine which has been shown to provide a good care plan/pathway for a range of patients under a variety of types of care. The protocols may deal with current patient conditions, past conditions, and/or may anticipate future outcomes for the patient. Hospitals typically have developed these based upon industry standards (e.g., the AARC) or through internal research. Protocols can deal with specific hardware settings, therapy use and medication (type, dosage, form of delivery, route to deliver) or can simply help guide choices in therapy based upon an evaluation.

### SUMMARY

In accordance with one embodiment of the disclosure, a method for configuring a protocol for use in providing a medical treatment to a patient is provided. A protocol may be selected for configuration, the protocol being associated with a medical treatment having a process flow including multiple steps. A protocol template may be created for the protocol, including selecting and arranging flowchart components to create a graphical flowchart representation of the process flow, and defining one or more variables for one or more steps in the process flow. The one or more variables may be linked to one or more existing objects such that when the protocol is executed, the existing objects are automatically accessed to facilitate execution of the protocol.

In accordance with another embodiment of the disclosure, a system for configuring a protocol for use in providing a medical treatment to a patient may be provided. The system may include software encoded in computer-readable media and operable to be executed by a processor. The software may provide a user interface for creating a protocol, the protocol being associated with a medical treatment having a process flow including multiple steps. The software may further provide a user interface for creating a protocol template for the protocol, including a user interface for selecting and arranging flowchart components to create a graphical flowchart representation of the process flow, and a user interface for defining one or more variables for one or more steps in the process flow. The software may further provide a user interface for linking the one or more variables to one or more existing objects such that when the protocol is executed, the existing objects are automatically accessed to facilitate execution of the protocol.

In accordance with another embodiment of the disclosure, a method for using a protocol for facilitating a medical treatment for a patient may be provided. The method may include initiating execution of a protocol, the protocol associated with a medical treatment and having a process flow including multiple steps, wherein a particular step has one or more associated variables linked to one or more existing objects. The method may further include executing the steps of the process flow, and during execution of the particular step, automatically accessing the existing objects linked to the one or more variables associated with the particular step in order to facilitate execution of the particular step.

In accordance with another embodiment of the disclosure, a system for configuring a protocol for use in providing a medical treatment to a patient may be provided. The system may include means for creating a protocol, the protocol being associated with a medical treatment having a process flow including multiple steps. The system may also include means for creating a protocol template for the protocol, including means for selecting and arranging flowchart components to create a graphical flowchart representation of the process flow, and means for defining one or more variables for one or more steps in the process flow. The system may also include means for linking the one or more variables to one or more existing objects such that when the protocol is executed, the existing objects are automatically accessed to facilitate execution of the protocol.

### BRIEF DESCRIPTION OF THE DRAWINGS

Some embodiments of the disclosure may be understood by referring, in part, to the following description and the accompanying drawings, in which like reference numbers refer to the same or like parts, and wherein:

FIG. 1 illustrates an example system for facilitating management of respiratory care according to one embodiment of the present disclosure;

FIG. 2 illustrates an example of various data communication pathways between users, software, and a hospital information system in a system such as shown in FIG. 1;

FIG. 3 illustrates a general method for configuring and implementing a medical protocol, according to one embodiment of the disclosure;

FIGS. 4-57 are example screenshots illustrating various aspects of configuring and implementing an example respiratory care protocol, according to one embodiment of the disclosure; and

FIGS. 58-61 illustrate additional example medical protocols that may be configured and implemented using any of the techniques discussed with respect to FIGS. 1-57.

### DETAILED DESCRIPTION OF THE DRAWINGS

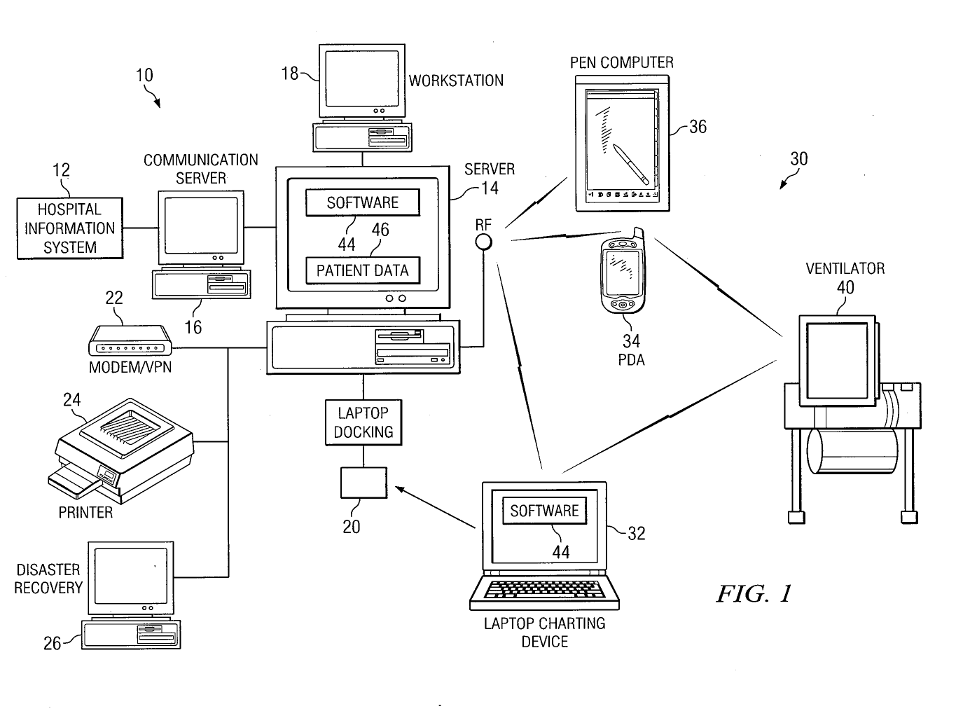

FIG. 1 illustrates an example system 10 for facilitating management of respiratory care according to one embodiment of the present disclosure. In some instances, system 10 may be associated with a care facility, such as a hospital, clinic, or retirement facility, for example. In this embodiment, system 10 may include a hospital information system (HIS) 12 that may communicate with a server 14 via zero, one, or more communication servers 16. Various system components, interfaces and/or peripherals may communicate with server 14, e.g., one or more workstations 18, docking stations 20, modem/VPN devices 22 (or any other suitable network interfaces), printers 24, and/or backup or recovery systems or databases 26.

Various user devices 30 may communicate data to and/or from server 14 via any suitable communication links, such as any suitable wireless or wireline links (e.g., RF links). User devices 30 may include one or more mobile devices, such as one or more laptops 32, PDAs 34, or other handheld computer devices 36, for example. User devices 30 may interface with one or more respiratory device 40 (e.g., one or more ventilators, CPAP, and/or BiPAP devices) in order to communicate data to and/or from such respiratory devices 40. User devices 30 may communicate with respiratory devices 40 via any suitable wireless or wireline communication links. In certain embodiments, a user device 30 may be carried by a user around the care facility as the user visits various patients or otherwise travels in or around the care facility, for example. One or more user devices 30 may be temporarily or permanently docked in docking stations 20 coupled to server 14, such as to charge the battery of a user device 30 and/or to communicate data between the user device and server 14.

As used herein, the term “user” may include any user of system 10, such as a respiratory therapist, a respiratory therapy (RT) director, a nurse, doctor, or other physician, for example. As used herein, the term “patient” may refer to any person or animal that may receive respiratory care from system 10, regardless of the medical status, official patient status, physical location, or any other characteristic of the person. Thus, for example, patients may include persons under official medical care (e.g., hospital patients), persons not under official medical care, persons receiving care at a medical care facility, persons receiving home care, etc.

Each component of system 10 may include any one or more suitable devices operable to accept input, process the input according to predefined rules, and/or produce output, for example, a personal computer, laptop, workstation, network computer, server, wireless data port, wireless telephone, personal digital assistant, one or more processors within these or other devices, or any other suitable processing device. Each component may include one or more processing units and/or memory units. Such processing units may be operable to execute software or other logic stored on one or more of such components, such as described below.

The various components of system 10 may form a network through which data may be exchanged or otherwise communicated. Such network may include, or be a associated with, any local area network (LAN), metropolitan area network (MAN), wide area network (WAN), wireless local area network (WLAN), virtual private network (VPN), intranet, Internet, any suitable wireless or wireline links, or any other appropriate architecture or system that facilitates communications in a network environment.

Hospital information system (HIS) 12 may include any typical HIS system, such as those known in the field, which system may store and or manage various data relating to the care facility, including data regarding patients. Communication server 16 may include any one or more computers operable to facilitate data communications between HIS 12 and server 14. Server 14 may manage the operation of system 10. In some embodiments, server 14 may store and/or execute respiratory care software 44, as well as storing patient data 46.

Respiratory care software 44 may be stored on one or more components of system 10. For example, in one embodiment, portions or modules of software 44 may be stored and executed at server 14, while one or more similar and/or different portions or modules of software 44 may be stored and executed at user devices 30. In other embodiments, software 44 may be stored and executed mainly or entirely at user devices 30.

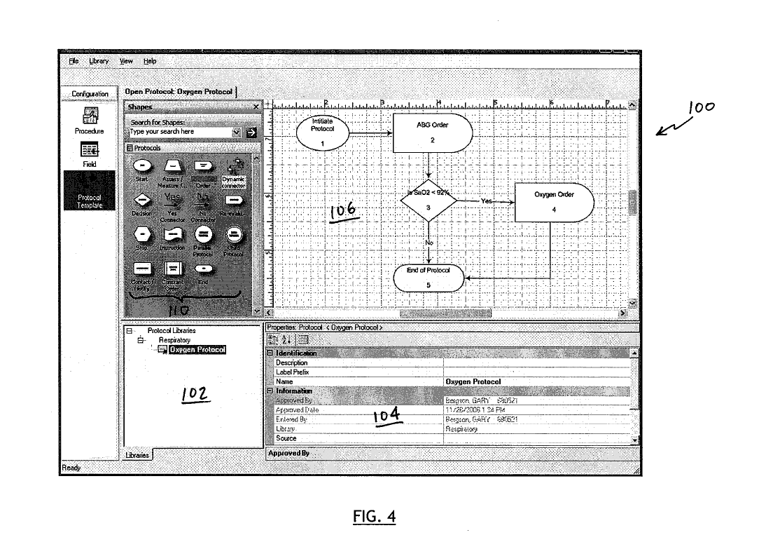

As in greater detail below with reference to FIGS. 4-57, software 44 may be protocol-based such that the software 44 may assist a user in following a particular protocol (e.g., a treatment plan) for administering respiratory care to a patient. As discussed herein, such protocols may be configurable such that a user (e.g., an RT director) may pre-configure any number of protocols and/or modify such protocols over time, as desired.

In operation, a user may carry a mobile user device 30 to a respiratory patient's room (e.g., “bedside”) and utilize respiratory care software 44 executable by the user device 30 in order to facilitate the management of respiratory care for the patient. For example, in some embodiments, the user device 30 may receive or download data from the patient's ventilator or other respiratory device 40. Alternatively or in addition, the user may enter into device 30 data from the ventilator screen and/or physiological data obtained by observing the patient or by taking measurements on the patient (e.g., using an oxymeter or other devices).

The data received by user device 30 may be analyzed by respiratory care software 44. As discussed above, software 44 may assist the user in administering respiratory care to the patient, such as by advising the user of particular orders or actions to be taken according to the particular protocol being executed. As a result, the user may be able to better manage respiratory care for a number of patients and/or increase the speed of care decisions, which may result in reduced recovery time, discontinuation of unnecessary or ineffective therapies, and/or other advantages. Further, by documenting the protocol activity and monitoring it actively, the system may also provide significant evidence to the effectiveness of the protocol and its implementation.

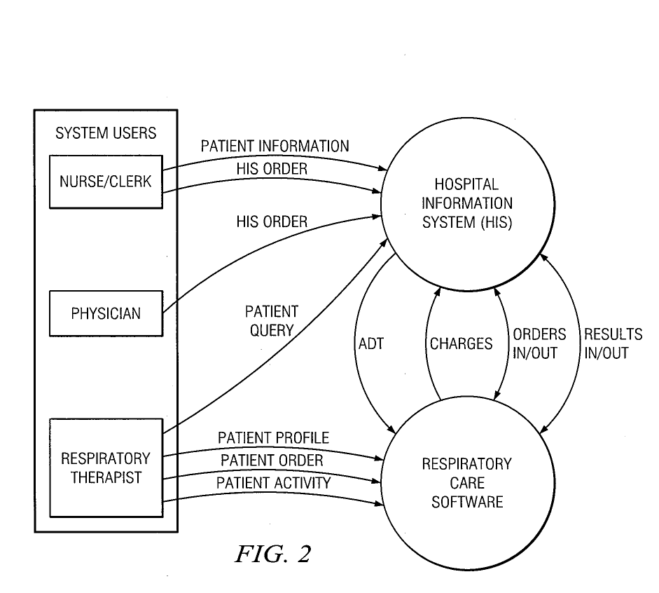

FIG. 2 illustrates an example of various data communication pathways between users, software 44, and HIS 12 in a system such as system 10. In this example, a nurse/clerk may communicate patient information and/or HIS orders to HIS 12. A physician may communicate HIS orders to HIS 12. A respiratory therapist may communicate patient information, patient orders, and patient activity data to HIS 12. HIS 12 may communicate ADT (admission, discharge, and transfers) data to software 44, and software 44 may communicate charges/billing data to HIS 12. In addition, HIS 12 and software may communicate orders and treatment results between each other.

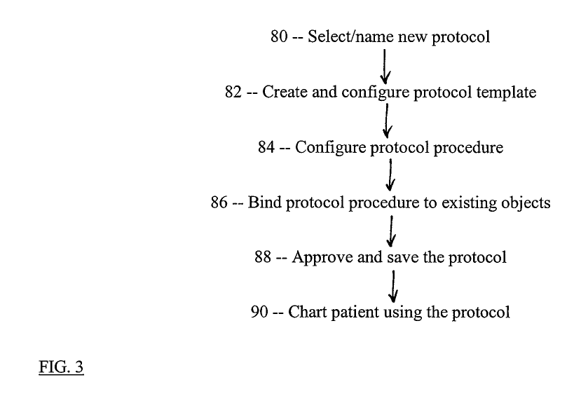

FIG. 3 illustrates a general method for configuring and implementing a medical protocol using software 44, according to one embodiment of the disclosure. At step 80, a user may interface with software 44 to select or name a new protocol to be configured. For example, a user may name a new protocol “O2 Protocol” or “Weaning Protocol” or “Prophylaxis Protocol.”

At step 82, the user may interface with software 44 to create and configure a protocol template. This may include building a flowchart representing a process flow for the protocol, which process flow may include, e.g., one or more orders, decisions, patient assessments or measurements, user instructions, etc. In some embodiments, software 44 may provide an interface allowing the user to create the flowchart by selecting, arranging, and connecting various flowchart components (e.g., shapes, connectors, etc.) in a workspace on the display screen. Creating and configuring the protocol template may also include defining one or more variables for one or more steps in the process flow. For example, for each step (or for certain steps), the user may enter information such as, e.g., a name of the step, formulas associated with the step (e.g., for decision steps), instructions or messages to be displayed in association with the step when the protocol is executed, etc. In some embodiments, once the protocol template is configured, the user may save, validate, and/or approve the protocol template. Validating the template may include software 44 determining whether the steps in the flowchart are properly arranged and/or connected such that the protocol may be appropriately executed.

At step 84, the user may interface with software 44 to configure a protocol procedure for the protocol. The user may first name a protocol procedure and tie the protocol procedure to the protocol template discussed above. The protocol procedure may include a protocol order definition including any number of order fields, and a protocol activity definition including any number of activity fields. The user may add any fields relevant to the protocol to the protocol order definition and/or to the protocol activity definition. One or more of such fields may be associated with existing objects, such as, e.g., an order or an activity for an existing procedure, or a field associated with an existing procedure (e.g., an order field or an activity field associated with an existing procedure). By adding fields that are associated with existing objects to the protocol order definition and/or to the protocol activity definition, the user may then tie such fields to particular steps in the process flow of the protocol, as discussed below regarding step 86.

At step 86, the user may interface with software 44 to bind, or link, the protocol procedure to one or more existing objects such that when the protocol is executed (e.g., at step 90), the existing objects are automatically accessed to facilitate execution of the protocol. For example, the process flow may include one or more decision steps having associated formulas that include formula variables (e.g., the formula “Is SaO₂<92?” includes the formula variable SaO₂). The user may link the each formula variable to one or more existing data fields such that when the formula step is executed (e.g., at step 90), data in the one or more existing data fields (e.g., an SaO₂ measurement) is automatically accessed and used for formula variables. As another example, the process flow may include one or more order steps, each having an associated order step name. The user may link each order step name to an existing order such that when that order step is executed (e.g., at step 90), data regarding the existing order (e.g., an order form and an activity form) is automatically accessed and displayed to the user for charting purposes.

At step 88, the user may interface with software 44 to approve and save the profile. The profile may now be available to be used (i.e., executed) for patient charting.

At step 90, a user (which may or may not be the same as the user that configured the profile) may interface with software 44 to chart a patient by executing the profile. The user may select the patient and initiate the protocol for the patient. Software 44 may then advance step by step through the process flow, including providing various instructions or suggestions to the user and prompting the user to enter various data (e.g., charting data, measurements, instrument readings, assessments, etc.) at various steps. The data entered by the user, as well as historical data regarding each step may be automatically stored as a record for subsequent access.

Configuring and Implementing an Example Protocol.

FIGS. 4-55 are example screenshots illustrating various aspects of software 44 related to configuring and implementing an example respiratory care protocol, according to one embodiment of the disclosure.

Building a Protocol Template

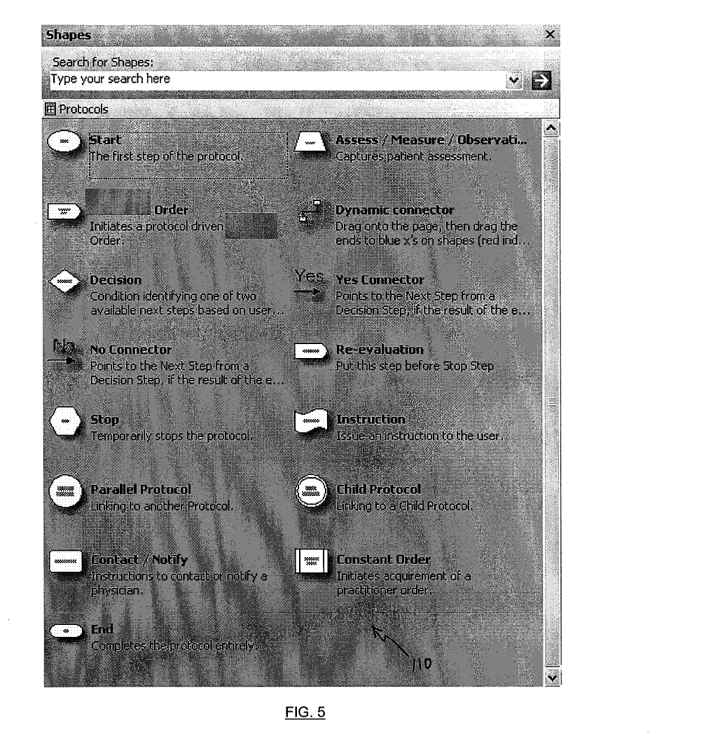

FIGS. 4-5: Workspace Background. FIG. 4 illustrates a display of a protocol template workspace 100 generated by an example embodiment of software 44. Protocol template workspace 100 may be provided for allowing a user to build a protocol template and may include multiple windows or regions, such as a library explorer region 102, a property control region 104, and a protocol designer region 106, for example. A protocol template may be defined as the branching logic for a process for treating a patient. The branching logic may comprise a flowchart including any number and/or variety of different steps linked together in any suitable manner.

Library explorer region 102 may generally be used for displaying protocol libraries, protocols, and/or other lists of items that may be selected by a user. Property control region 104 may generally be used for displaying and allowing user entry of various properties (e.g., names, variables, formulas, values, etc.) regarding various components of the protocol template. For example, property control region 104 may display and allow a user to edit various properties associated with a formula-based decision step of a protocol template being configured by the user. Protocol designer region 106 may generally be used for displaying and allowing a user to construct a graphical representation of a protocol template by assembling and connecting various shapes 110 corresponding to various flowchart steps.

FIG. 5 illustrates an example set of available shapes 110 that may be selected by a user for constructing a protocol template, according to one embodiment of the disclosure. Each shape 110 corresponds with a predefined type of protocol step. In this embodiment, the set of available shapes 110 includes the following:
Start: The first step of the protocol
Assess/Measure/Observation: Captures patient assessment
Order: Initiates a protocol-driven order
Decision: Condition identifying one of two available next steps based on user-defined data
Dynamic Connector: Connects two consecutive steps
Yes Connector: Points to next step from a Decision step, if the result of the evaluation is “Yes”
No Connector: Points to next step from a Decision step, if the result of the evaluation is “No”
Re-evaluation: Initiates a re-evaluation; inserted before a Stop step
Stop: Temporarily stops the protocol
Instruction: Issues an instruction to the user
Parallel Protocol: Links to another protocol
Child Protocol: Links to a child protocol
Contact/Notify: Instructions to contact or notify a physician
Constant order: Initiates acquiring of a practitioner order
End: The last step of the protocol

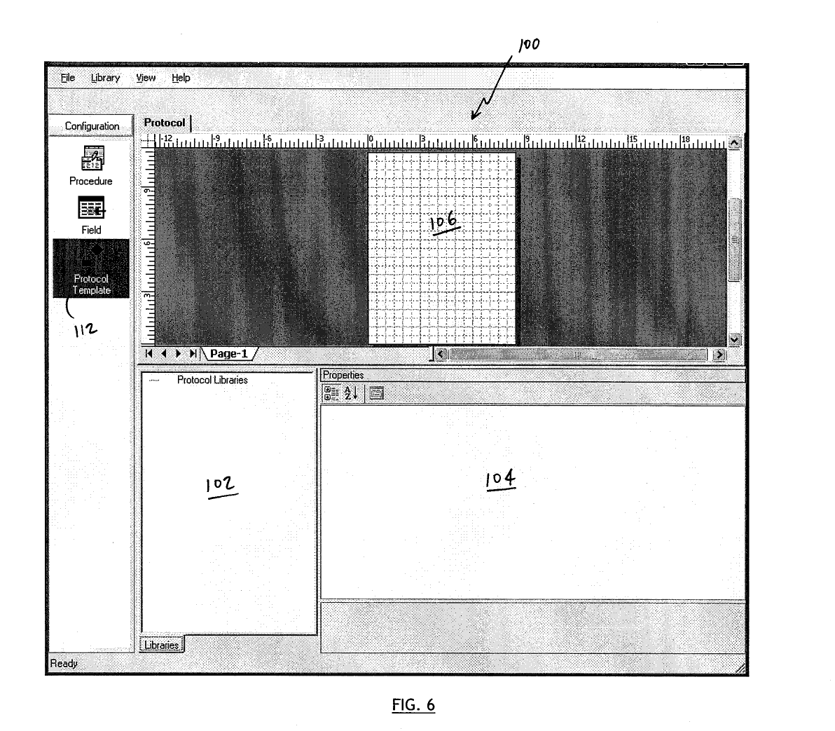

FIG. 6: Accessing the Protocols module. To begin building a protocol template, the user may select the “Protocols Template” icon 112 from various icons listed under a “Configuration” menu. This selection opens a blank protocol template workspace 100, including a listing of existing Protocol Libraries in library explorer region 102, each of which may contain one or more protocol. No existing protocol libraries are shown in this example.

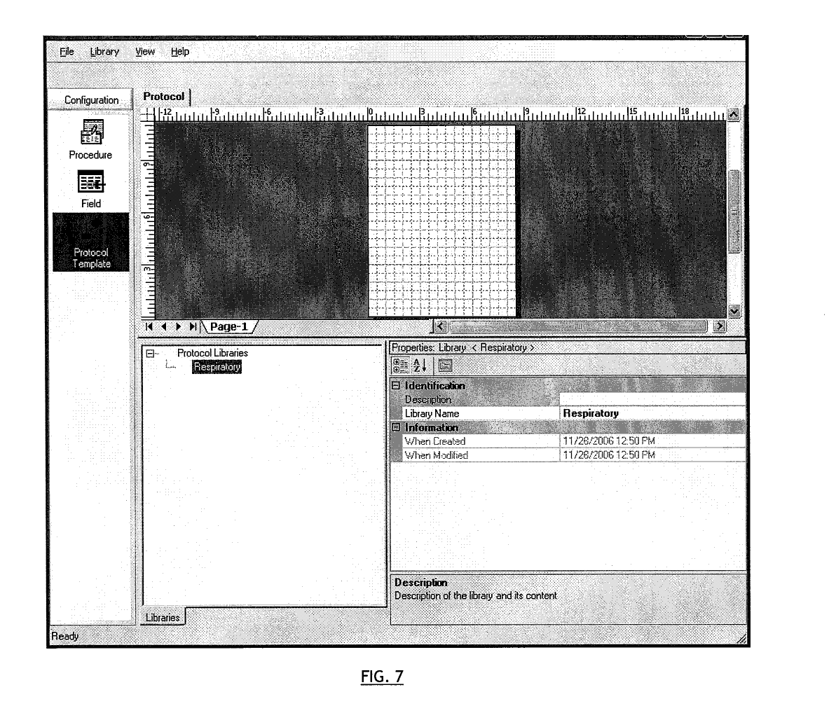

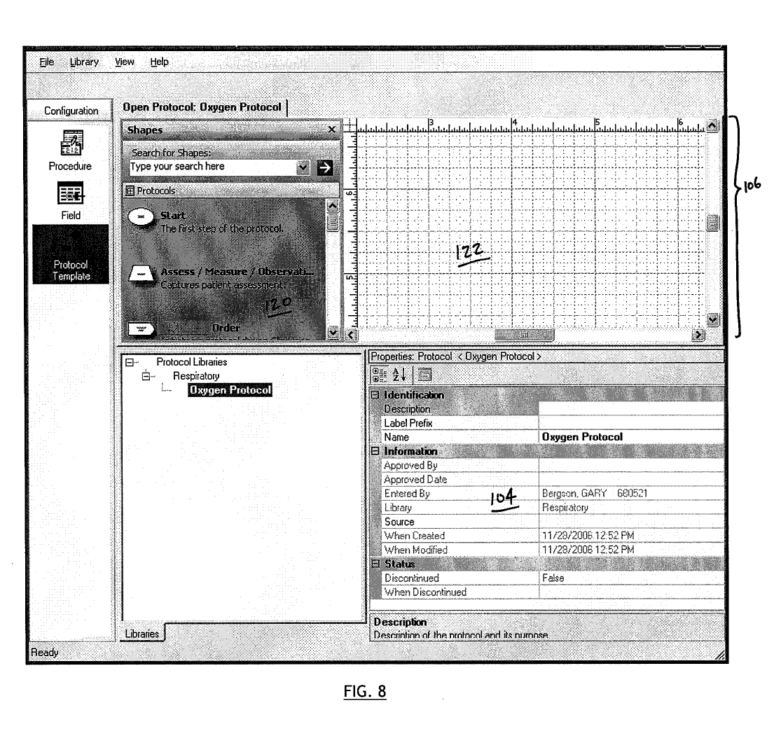

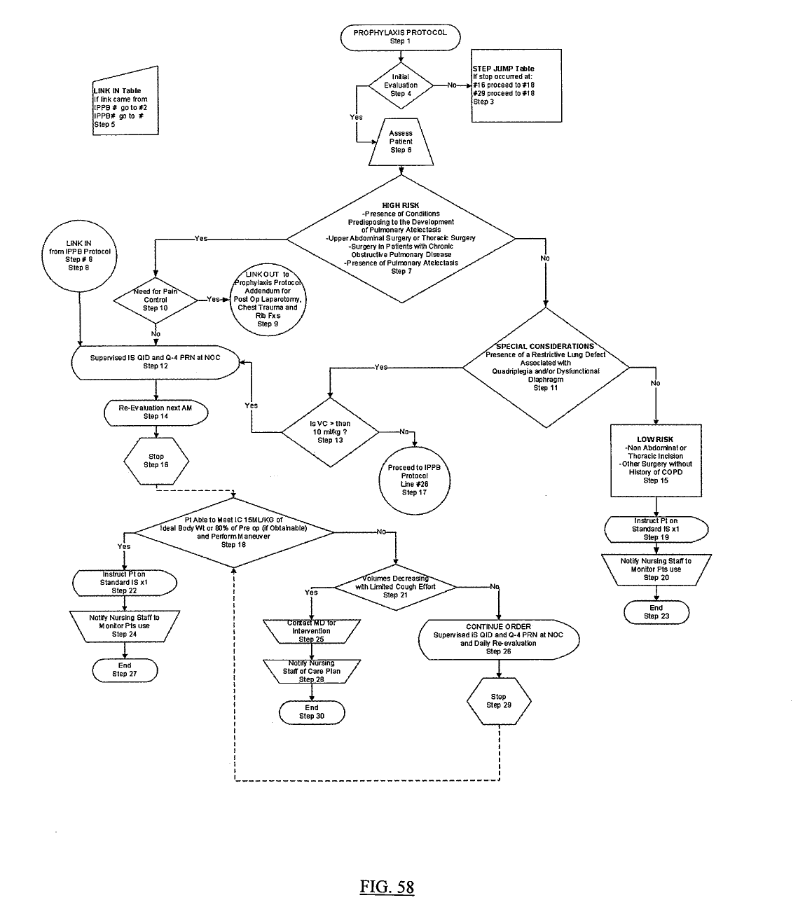

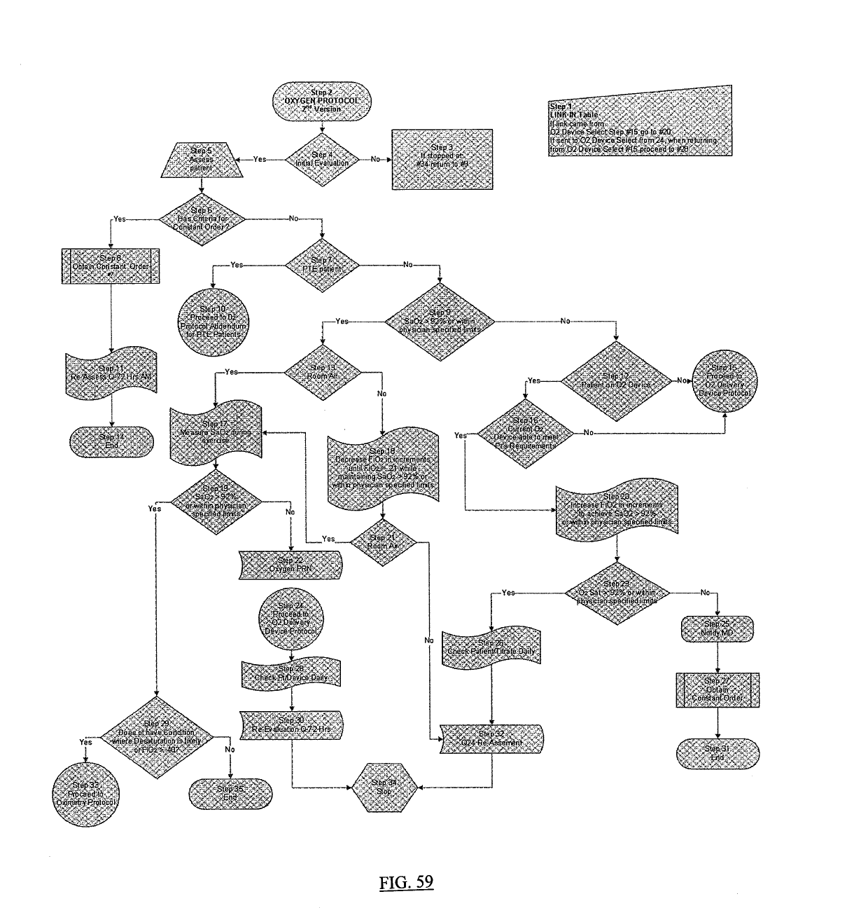

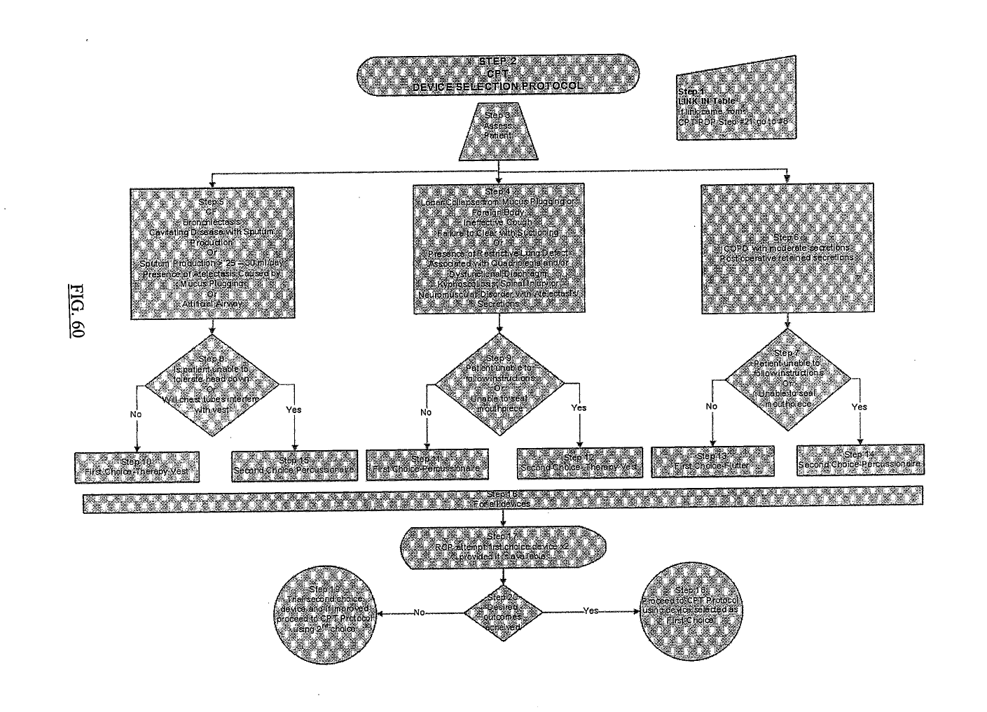

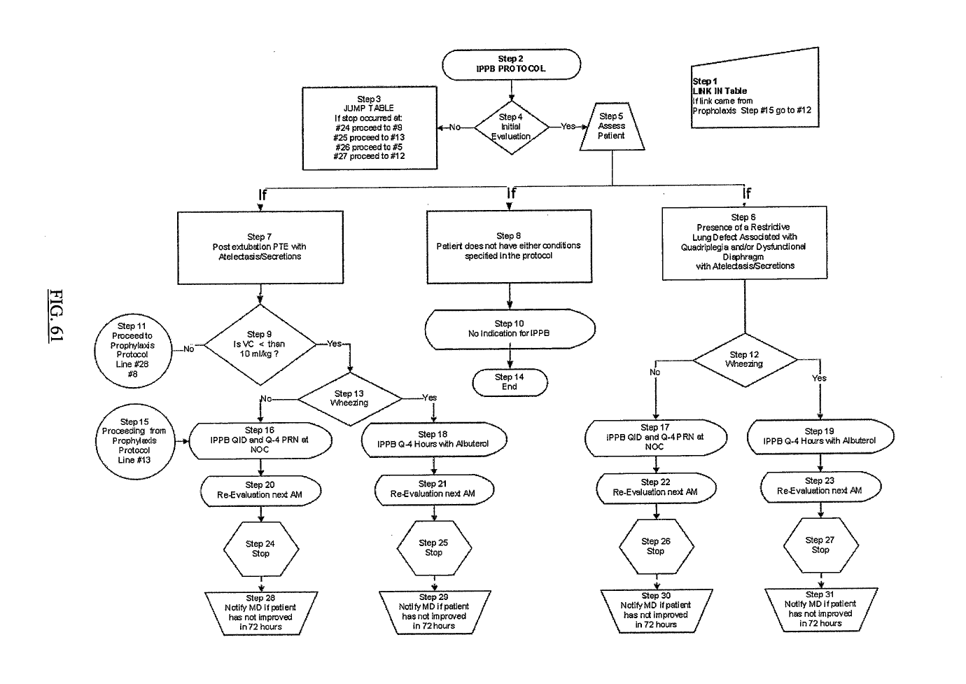

FIGS. 7-8: Selecting an existing protocol or naming a new protocol. The user may either select an existing protocol from an existing protocol library to access (e.g., to review or modify) or may create a new protocol. In one embodiment, an existing protocol may be selected by clicking and dragging the protocol name into region 106. In some embodiments, one or more protocols may be pre-loaded onto software 44, such as one or more protocols used by AARC guidelines, for example. In other embodiments, no protocols are pre-loaded onto software 44; in such embodiment, the user (e.g., RT director) may create/build the desired protocols, e.g., as described herein. Example protocols, as well as instructions for creating/configuring such protocols using the systems/software of the present disclosure, are shown in FIGS. 58-61.

In this example, the user creates a new protocol. To create a new protocol, the user may click on “Protocol Libraries,” then “Create Library,” and then type in the name of a new protocol library: “Respiratory” (see FIG. 7). The user may then click on the newly created “Respiratory” library, then “Create Protocol,” and then type in the name of a new protocol: “Oxygen Protocol” (see FIG. 8). When the name of the new protocol is entered, various protocol parameters may be displayed in regions 104 and 106 relative to the newly created Oxygen Protocol.

As discussed above, protocol designer region 106 generally displays a graphical representation of the process flow of the protocol template for the selected protocol (here, Oxygen Protocol). In some embodiments, region 106 may allow a user to select from multiple or alternative views of the process flow for the protocol template. For example, region 106 may include (a) an “Explore” tab that may be selected to show a “true view” of the process flow, and (b) a “Flowchart” tab that may be selected to show a graphical “flowchart view” of the process flow, such as a Visio-like view for example. The user may select between the multiple views as desired, as different users may be more comfortable with one view than another. In some embodiments, software 44 may include relevant portions of VISIO™ or a similar flowchart-oriented software. In the illustrated embodiment, only the graphical “flowchart view” of the process flow is illustrated in region 106. As shown in FIG. 8, protocol designer region 106 may include multiple sub-views, such as a shapes sub-view 120 and a template layout sub-view 122, for example.

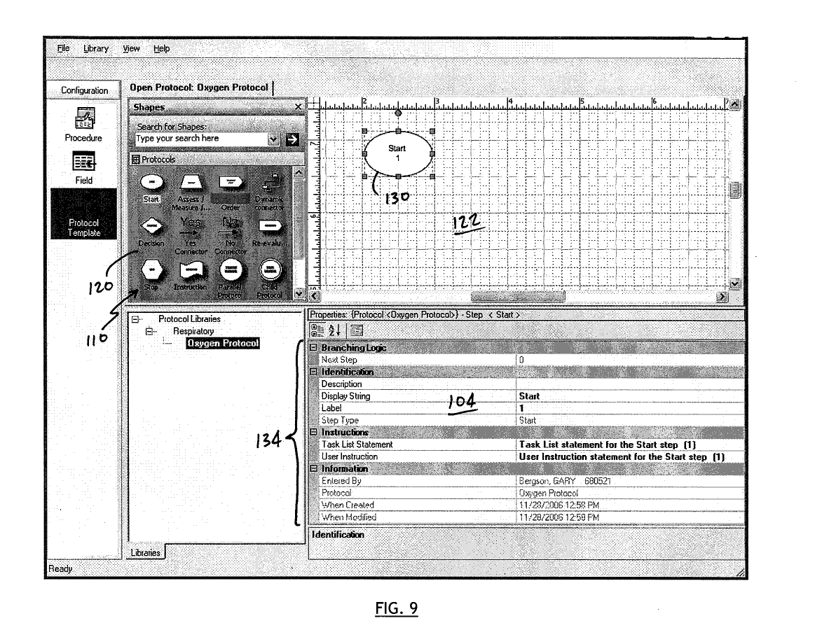

FIGS. 9-19: Creating the process flow. Once the new protocol has been created, the user may build and define the process flow of the protocol template. Building and defining the process flow includes arranging and connecting shapes 110 as desired to create a process flow in template layout sub-view 122, and entering various information regarding each step of the process flow. In some embodiments, these two tasks may be performed in any order. For example, the user may arrange and connect shapes 110 to form the complete process flow, and then enter relevant information for each step in the process flow. As another example, the user may enter relevant information for each step after adding that step to the process flow.

To arrange and connect shapes 110 to form the process flow, the user may drag and drop shapes 110 from shape sub-view 120 into template layout sub-view 122 as desired. The user may use a paper or other copy of the desired protocol as a guide for selecting and arranging the appropriate shapes 110.

In this example, as shown in FIG. 9, the user may drag a “Start” shape 130 into sub-view 122. In response, property fields 134 corresponding to the Start step are displayed in region 104. As shown in FIG. 9, some or all property fields 134 may be auto-populated with default information. Different types of steps may have different corresponding property fields 134. For example, a “Decision” step may include a “formula” property field, whereas a “Start” step or an “Order” step may not. The property fields for each step may form a data table for that step. Property fields may be classified under a number of property categories, including, for example:

Binding Information properties: define variables that may be bound to existing objects, e.g., existing orders and/or various existing fields. For example, for an Order step, binding information properties may include a “Procedure Name” field, which may be used to bind the Order step to an existing order. As another example, for a Decision step, binding information properties may include a “Formula” field, which may be used to enter a formula that includes one or more variables that may be used to bind the formula to one or more existing fields.

Branching Logic properties: define logical relationships between that step and one or more other steps. Branching logic fields may include, for example, “NextStep” and “AltenativeNextStep” fields. As the various steps are linked using connecting shapes 110 (e.g., Yes, No, and Dynamic connectors), one or more of the branching logic fields may auto-populate according to the arrangement of the flowchart. In some embodiments, the “Yes” decision leading from a particular decision step is listed as the NextStep for the particular decision step, and the “No” decision leading from the particular decision step is listed as the AlternativeNextStep for the particular decision step. The relationships created between the various steps by using the connecting shapes 110 direct the system how to behave (i.e., how to proceed through the process flow) once the protocol template is implemented.

In some embodiments, branching logic properties for certain types of steps (e.g., decision steps) may include a “User Confirmation Required” field, which (if set to True) may allow a user to confirm or override particular results provided by software 44. For example, a decision step “User Confirmation Required” field, which (if set to True) may allow a user to confirm or override an automatic decision of software 44. Such user confirmation feature may be useful, for example, in situations where the user has additional knowledge about the patient, treatment, etc. that may not be factored into the automated decision.

Identification properties: includes various information to identify the particular step, e.g., a Description, a Display String (that is displayed in the flowchart in sub-view 122), a Label, and a Step Type.

Instructions properties: define instructions that may be displayed in connection with that step during run-time execution of the protocol (i.e., during patient charting using the protocol). Example instruction fields may include “Task List Statement” and “User Instruction” fields. Task List Statements may later appear as action items in a list to be checked off by a user (e.g., therapist) during patient charting (as discussed below). For example, Task List Statements may include “Oximetry Protocol started” or “Assessed Patient.” User Instructions may later appear as instructions or suggestions to a user (e.g., therapist). For example, User Instructions may include “Begin Protocol” or “Enter Assessment Data.” In some instances, multiple instructions may be entered for a single step.

Information properties: includes various information regarding the step, e.g., the name of the user who entered the step, the protocol in which the step is included, the date and time when the step was created, and the date and time when the step was last modified.

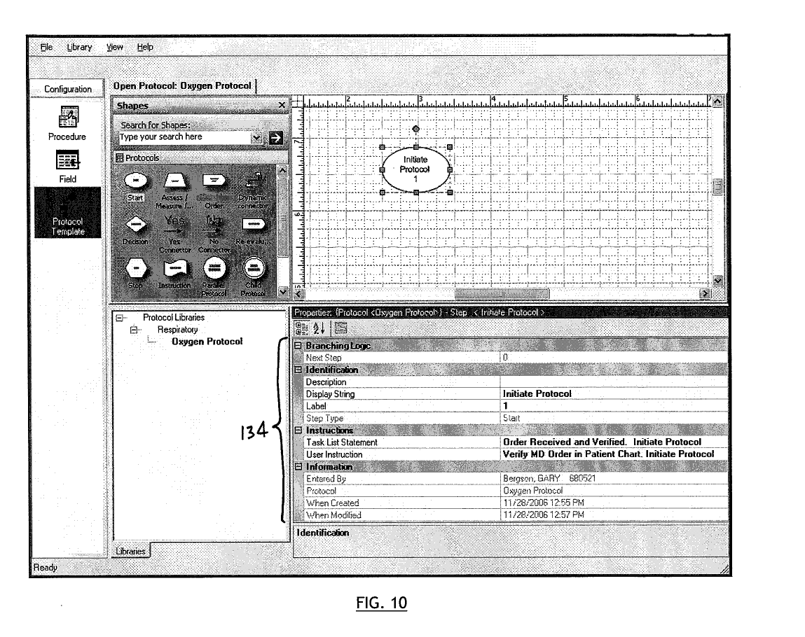

As shown in FIG. 10, the user may edit the data in particular property fields 134 regarding the Start step from the default text/values to any desired text/values.

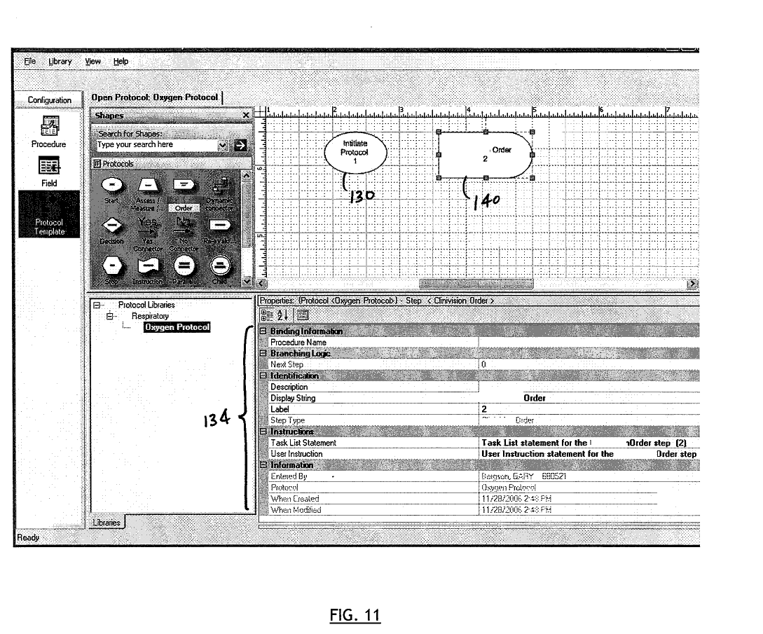

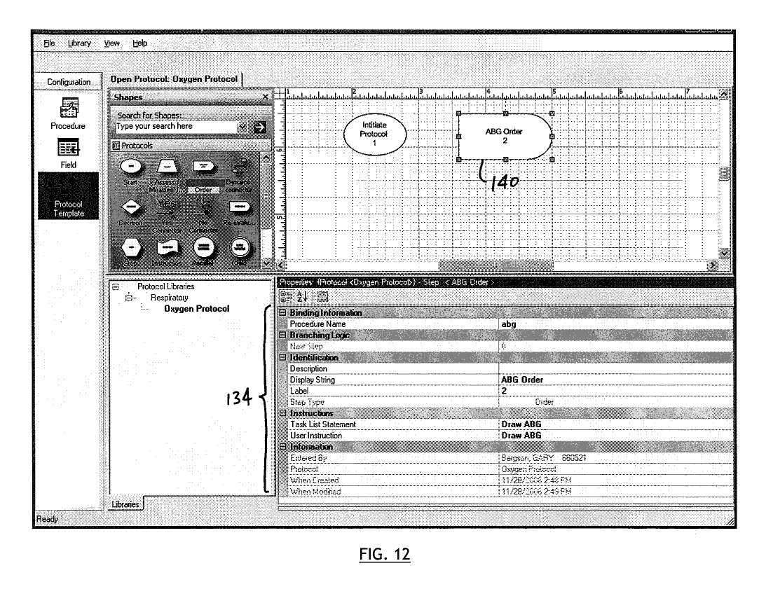

As shown in FIG. 11, the user may then drag an “Order” shape 140 into sub-view 122. In response, property fields 134 corresponding to the Order step are displayed in region 104. Some or all property fields 134 may be auto-populated with default information corresponding to an Order step. As shown in FIG. 12, the user may edit the data in particular property fields 134 regarding the Order step from the default text/values to any desired text/values. For example, the user enters the Procedure Name “abg” and edits the Display String to “ABG Order.”

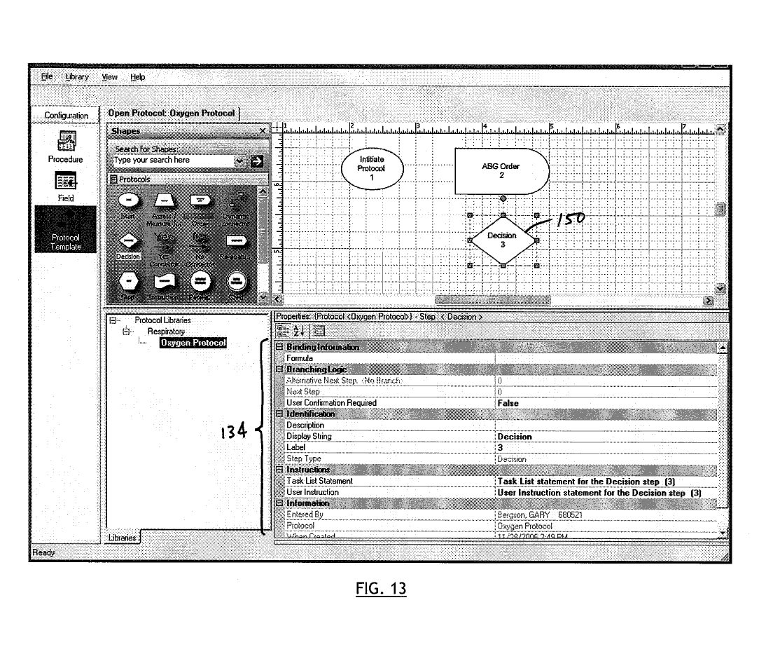

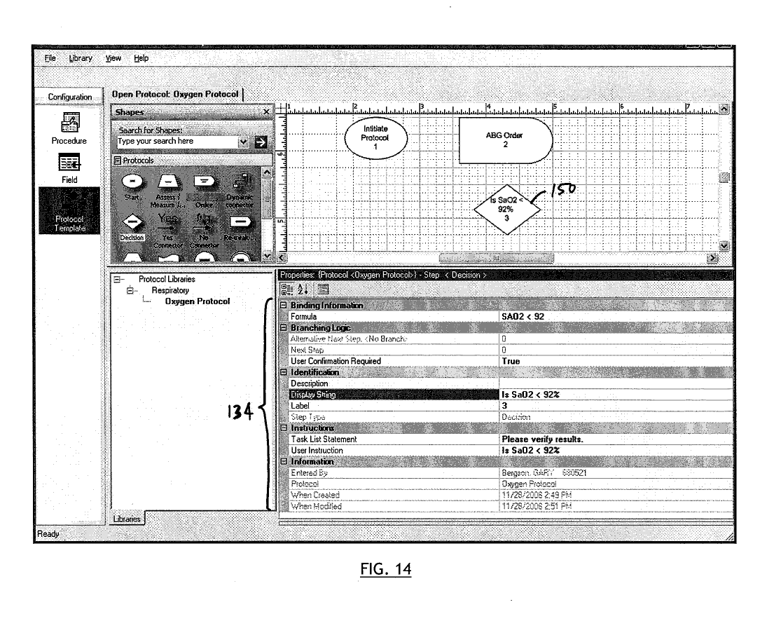

As shown in FIG. 13, the user may then drag a “Decision” shape 150 into sub-view 122. In response, property fields 134 corresponding to the Decision step are displayed in region 104. Some or all property fields 134 may be auto-populated with default information corresponding to a Decision step. As shown in FIG. 14, the user may edit the data in particular property fields 134 regarding the Decision step from the default text/values to any desired text/values.

In particular, the Decision step may include a “Formula” field, and a “User Confirmation Required” field. The “Formula” field is used for entering the formula by which the decision is made at the Decision step. In this example, the user enters the formula “SaO₂<92” such that when the protocol is executed and the Decision step is reached, the software will retrieve the patient's SaO₂ level data and determine whether it is less than 92 percent. Other example formulas include:
ConstantOrder=“yes”
IsSpecialProc=“yes”
FiO2<40
PIP>40
Heart Rate>120

The setting for the “User Confirmation Required” field determines whether to require the user to confirm the result of the decision (i.e., whether to advance along the Yes or No connector paths to the next step according to the result of the Decision step) during run-time (i.e., when charting a patient using the protocol). The field may be set to True or False. In this embodiment, the default value is False (see FIG. 13) and the user has changed the setting to True (see FIG. 14). If the field is set to False, the protocol will automatically advance to the next step along either the Yes or No path based on the result of the Decision. If the field is set to True, when a decision is made at the Decision step during run-time, the software will prompt the user (e.g., therapist) to confirm the decision. For example, in the present example, a message may be displayed reading “SaO₂<92=True. Do you accept this?”

If the user confirms the decision, the protocol will advance according to the decision. If not, the system may prompt the user to enter a new value (in a dialog box), direct the protocol to advance along the other path (e.g., along the No path after a Yes decision), allow the user exit from the protocol, or take some other action. Thus, the user confirmation feature allows the user to override the automated decision provided by the software, which may be useful, for example, in situations where the user has additional knowledge about the patient, treatment, etc. that may not be factored into the automated decision.

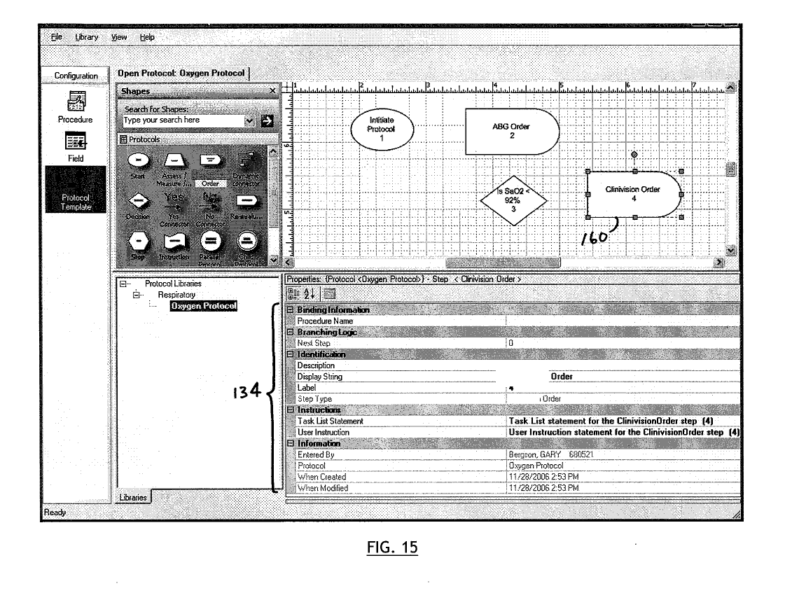

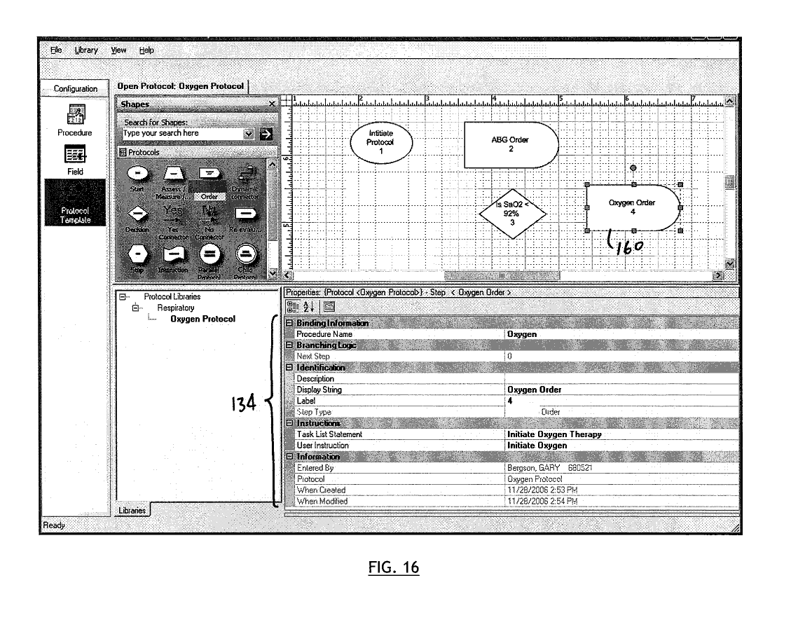

As shown in FIG. 15, the user may then drag another “Order” shape 160 into sub-view 122. In response, property fields 134 corresponding to the Order step are displayed in region 104. Some or all property fields 134 may be auto-populated with default information corresponding to an Order step. As shown in FIG. 16, the user may edit the data in particular property fields 134 regarding the Order step from the default text/values to any desired text/values. For example, the user enters the Procedure Name “Oxygen” and edits the Display String to “Oxygen Order.”

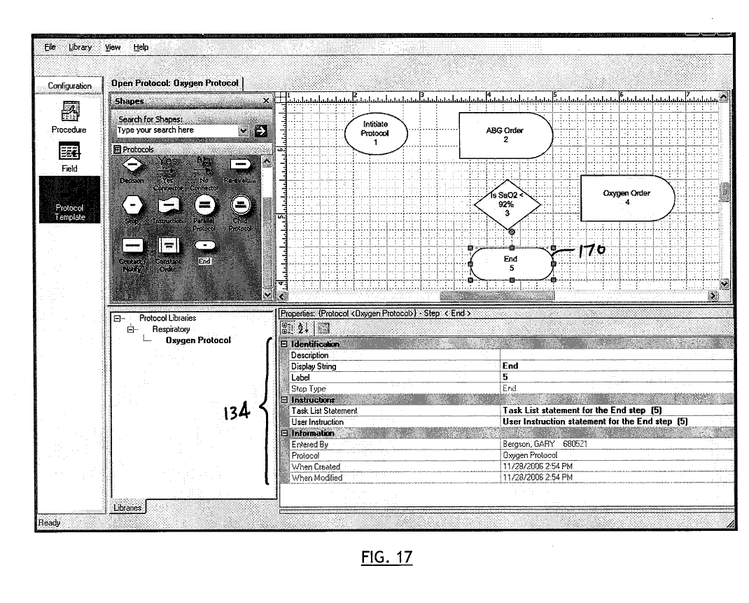

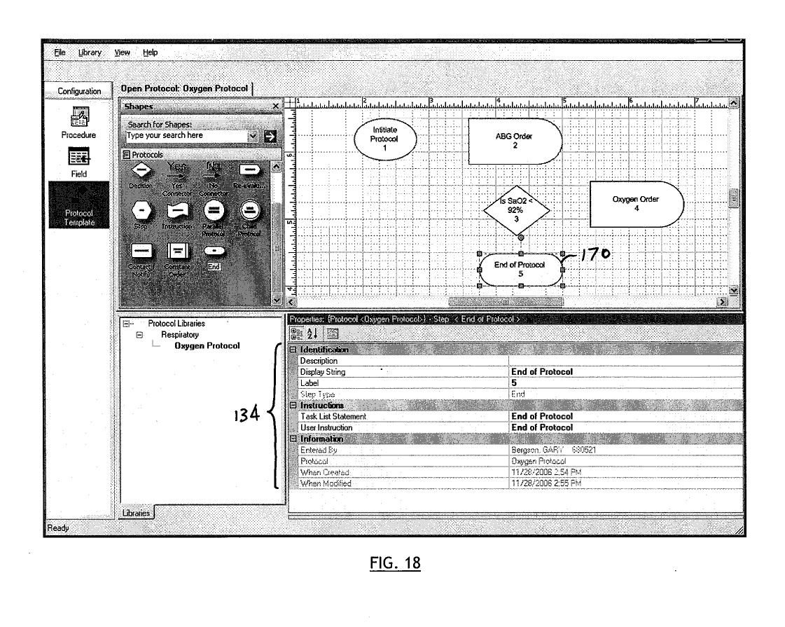

As shown in FIG. 17, the user may then drag an “End” shape 170 into sub-view 122. In response, property fields 134 corresponding to the End step are displayed in region 104. Some or all property fields 134 may be auto-populated with default information corresponding to an End step. As shown in FIG. 18, the user may edit the data in particular property fields 134 regarding the End step from the default text/values to any desired text/values.

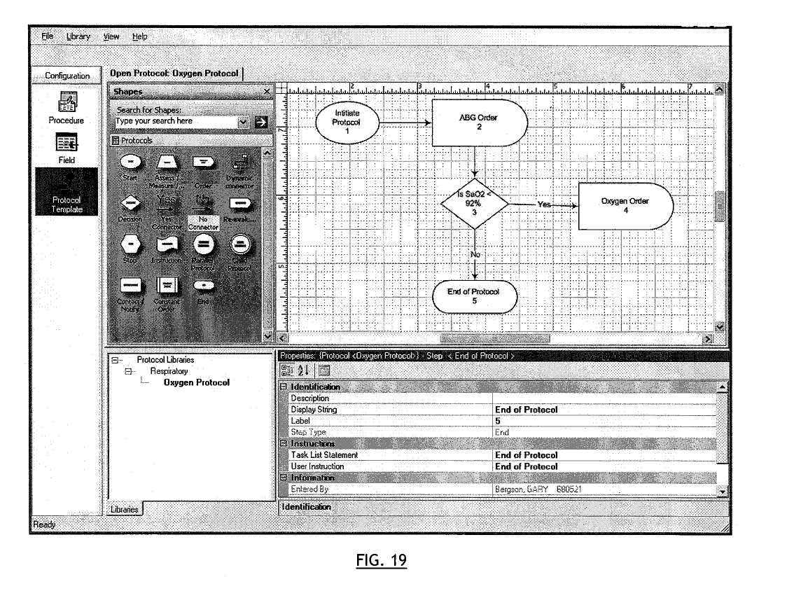

As shown in FIG. 19, the user may then connect steps 1-5 by dragging connectors (e.g., Yes, No, and Dynamic connectors) from sub-view 120 to sub-view 122. In this example, the user drags a Dynamic connector to connect steps 1 and 2, and to connect steps 2 and 3. The user also drags a Yes connector and a No connector to connect Decision step 3 to steps 4 and 5, respectively. In some embodiments, the Dynamic connector may be manipulated by the user to bend or turn (e.g., 90-degree turns) as desired to create the flowchart. As the various step/decision icons 140 are linked using connecting icons 144, the “Properties” display in a property control region 104 may begin to auto-populate to indicate the relation between steps in the flowchart.

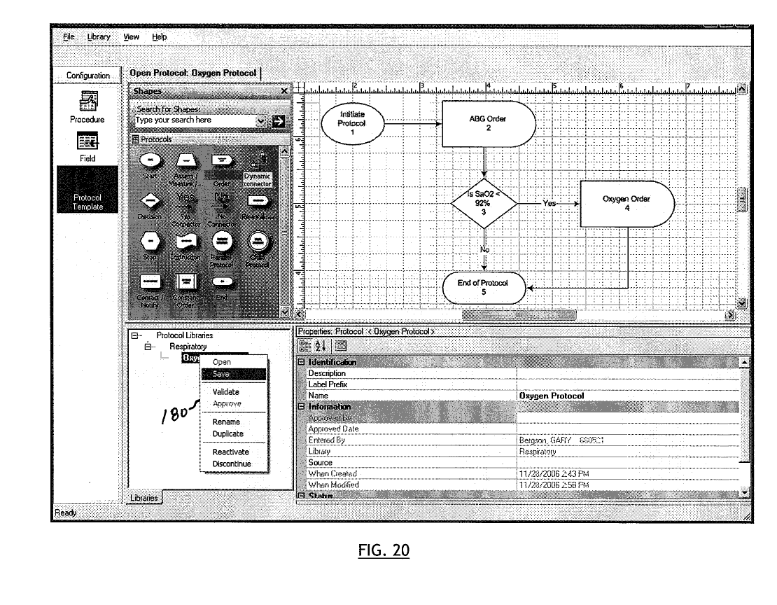

FIGS. 20-23: Saving, Validating, and Approving the Protocol Template. As shown in FIG. 20, the user may save the protocol template, e.g., by clicking “Save” from an options menu 180.

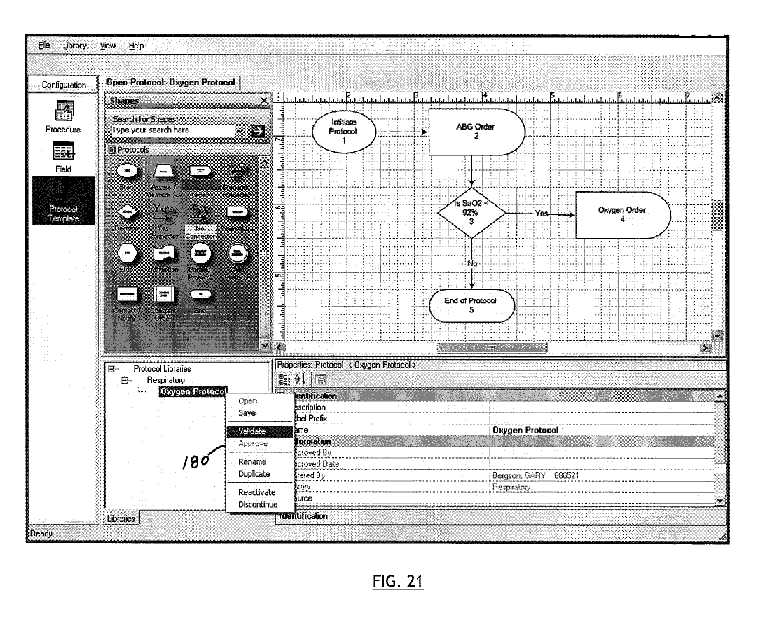

As shown in FIG. 21, the user may then validate the protocol template, e.g., by clicking “Validate” from options menu 180. In some embodiments, the validation feature may check whether the steps are appropriately connected by connectors (e.g., Yes, No, and Dynamic connectors), whether any mandatory fields have been filled, etc. If the protocol template is not validated (e.g., if there is no connector from step 4 to step 5), the software may display a message (e.g., a pop-up window) notifying the user of the invalid aspect. The user may then correct the invalid aspect and re-validate. If the protocol template is validated, the software may display a message (e.g., a pop-up window) notifying the user that the protocol template has been validated. The protocol template may now be bound, e.g., as discussed below.

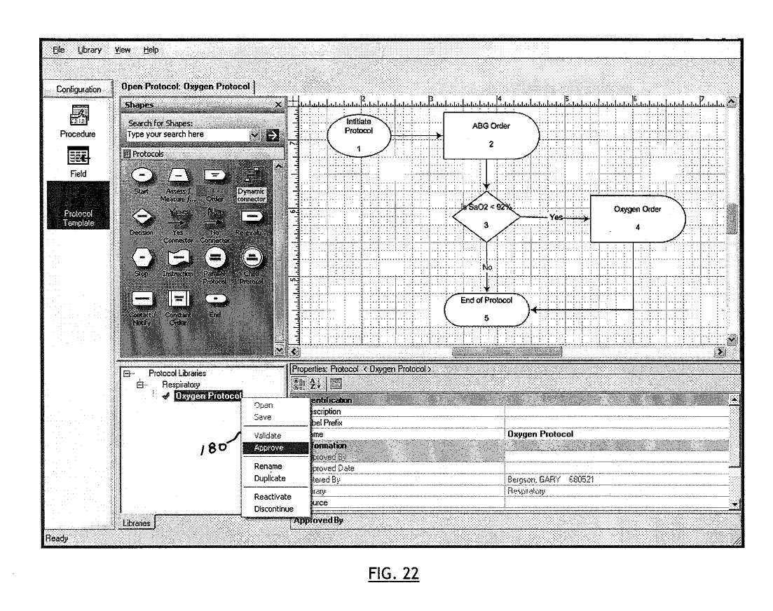

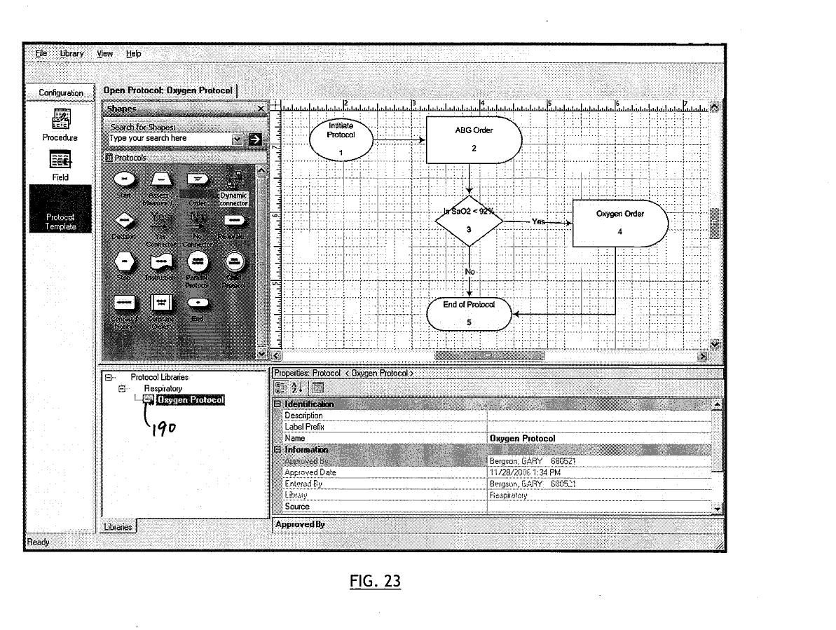

As shown in FIG. 22, the user may approve the protocol template, e.g., by clicking “Approve” from options menu 180. In some embodiments, once the protocol template has been approved by the user, an approval icon 190 is displayed to indicate that the protocol template has been approved, as shown in FIG. 23.

Other users may also approved the protocol template over time, and the software may maintain a record of all users who have approved the protocol template. Subsequent users may access this information to determine which users and/or how many users have approved the particular protocol template, which may provide an indication of the reliability or other measure of the protocol template.

In this manner, a user may select a protocol or created a new protocol, create a process flow for protocol template, define the properties for each step in the protocol template, and validate and approve the protocol template. The user may then create a protocol procedure definition and bind it to the protocol template, e.g., as discussed below.

Creating a Protocol Procedure Definition and Binding to the Protocol Template

Procedures, Orders, and Activities. Software 44 may maintain or have access to a number of “procedures,” which may either be preloaded or user-configured. A procedure may include (a) an order and (b) any activities resulting from that order. For example, an Aspirin procedure may include (a) an order: take aspirin every two hours, and (b) activities: the actual taking of aspirin every two hours.

In the medical field, an order refers to an medical procedure or action to be performed by a physician or other medical personnel for a patient, e.g., “Start an oxygen prn,” “Perform a sat,” “Perform a blood gas,” “Take aspirin,” etc. Orders may come in to the respiratory care system throughout the day (e.g., orders that a doctor has given for a patient in ER or after surgery). For example, orders may enter into the respiratory care system from the hospital's information system (HIS).

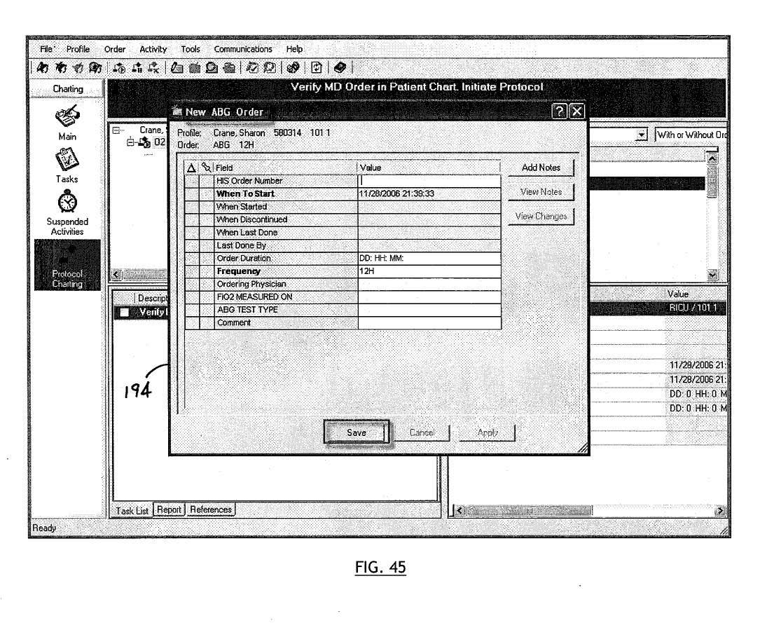

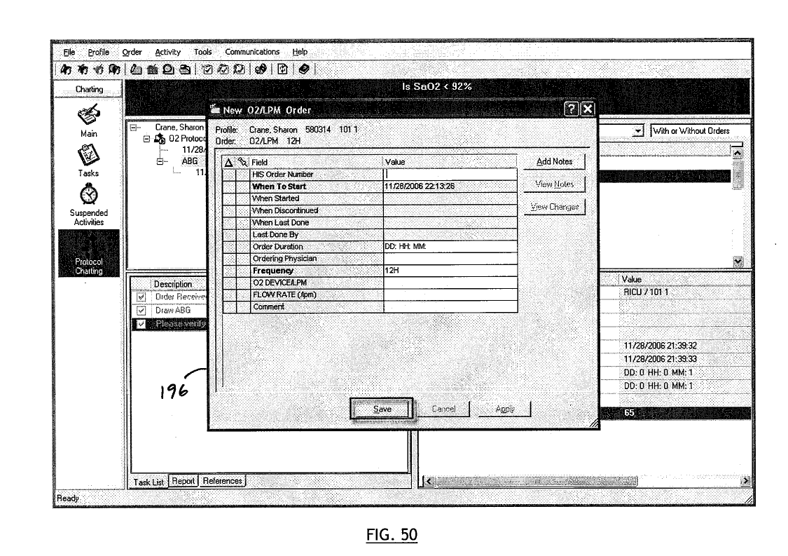

In the context of software 44, an order refers to the set of data defining various parameters of a medical order. An order may have an associated order form that includes a group of order fields including data regarding the order. Example order fields may include, e.g., the order number, when to start the order, when the order was started, when the order was ended, the order duration, the frequency of the order (e.g., every 12 hours), the ordering physician, and/or various settings for a medical device (e.g., pressure or flow rate settings for a ventilator). The order form may be used for generating a record of the details of an order to be carried out for a patient. In some embodiments, the data/values for certain order fields may be automatically populated by software 44, while the data/values for other order fields may be entered by a user (e.g., a therapist carrying out the order on the patient). Example order forms, namely an ABG order form 194 and an O2/LPM order from 196, are shown in FIGS. 45 and 50, which are discussed below in greater detail. In these example, the grayed fields are automatically populated with data by software 44, while the non-grayed fields may be entered by a user.

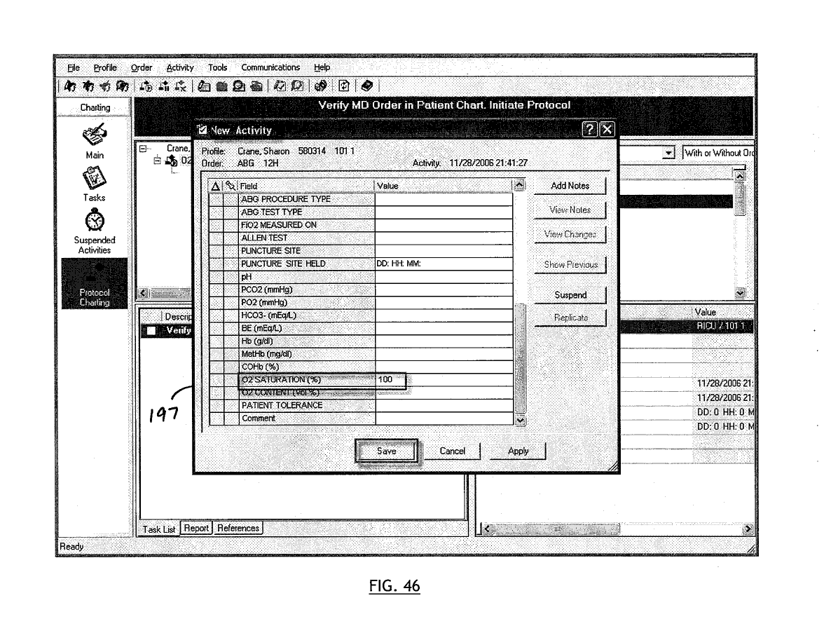

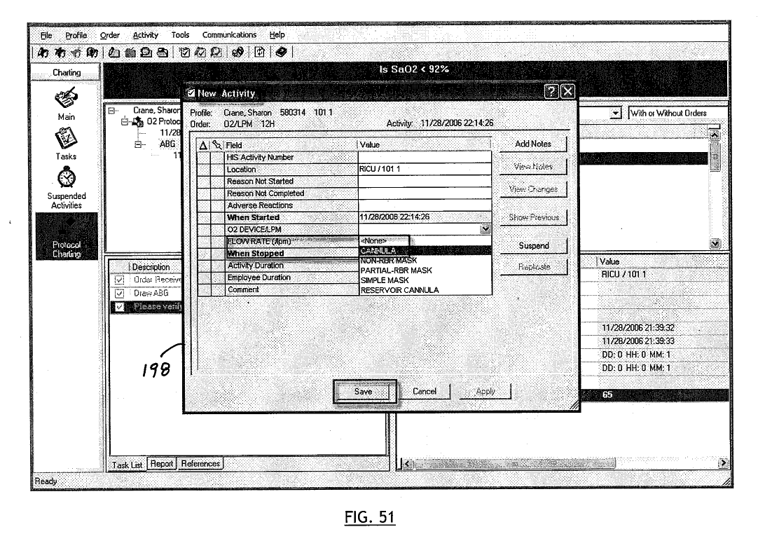

Similarly, an activity may have an associated activity form that includes a group of activity fields including data regarding activities for an order. Example activity fields may include, e.g., activity location, when the activity was started, when the activity was ended, activity duration, employee duration, reason not started, reason not completed, and/or various data regarding actions, tests, procedures, etc. performed by a user. The activity form may be used for generating records of the details of activities performed for a patient as a result of an order. In some embodiments, the data/values for certain activity fields may be automatically populated by software 44, while the data/values for other activity fields may be entered by a user (e.g., a therapist carrying out the activity on the patient). Example activity forms, namely an ABG activity form 197 and an O2/LPM activity from 198, are shown in FIGS. 46 and 51, which are discussed below in greater detail. In these example, the grayed fields are automatically populated with data by software 44, while the non-grayed fields may be entered by a user.

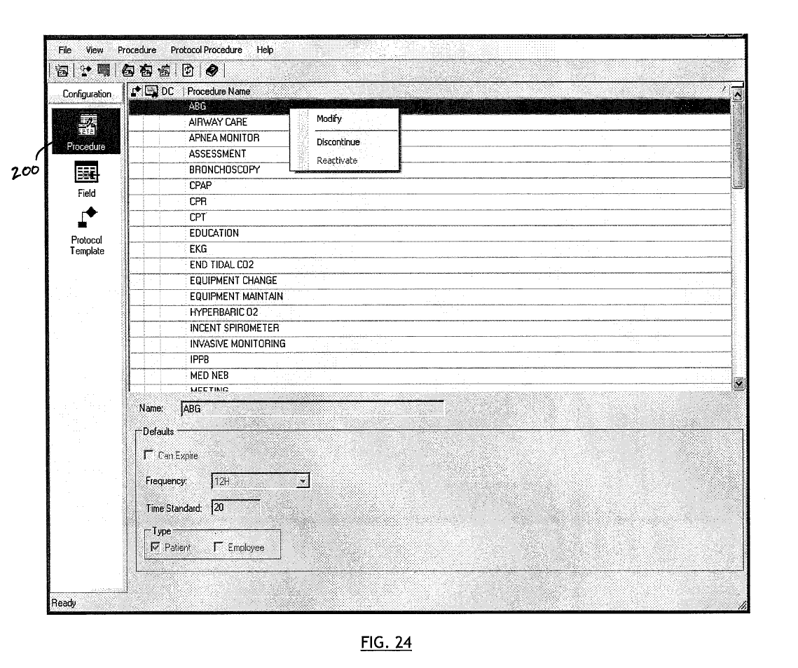

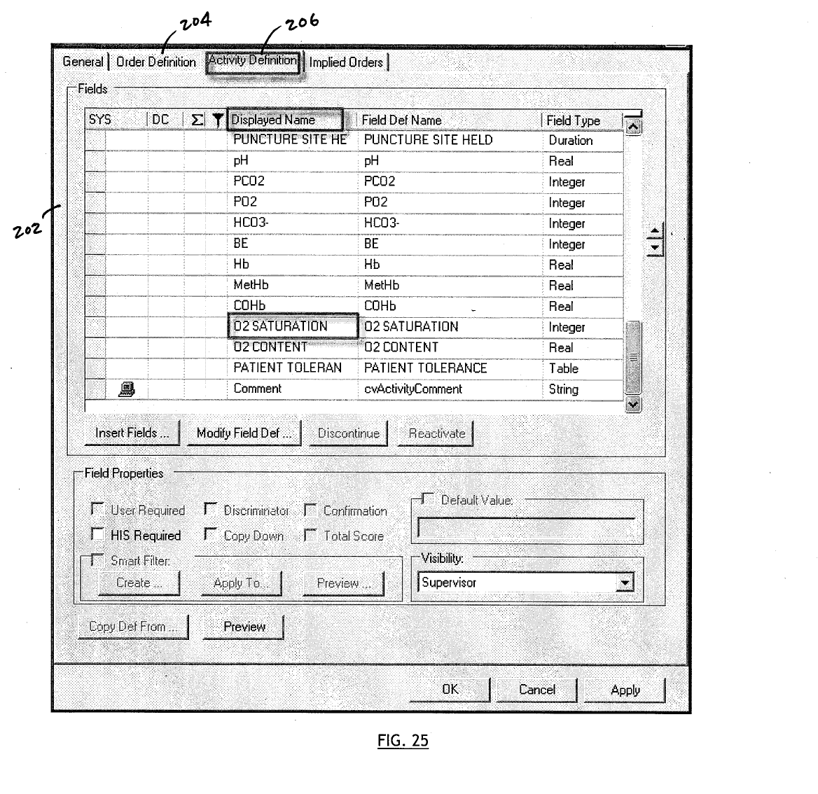

The order fields included in an order form and the activity fields included in an activity form may be defined by an “order definition” and an “activity definition,” which are components of a “procedure definition” for a particular procedure. At least a portion of the fields included in an order definition and an activity definition for a particular procedure may be selected and customized by a user, as discussed below with respect to FIGS. 24 and 25.

Software 44 may maintain or have access to multiple procedures, each of which generally corresponds to a medical order. For example, software 44 may maintain or have access to an ABG Procedure (which includes an ABG order and ABG activities), an Oxygen Procedure (which includes an Oxygen order and Oxygen activities), etc.

Separate from these procedures, a protocol procedure (in this example, an Oxygen protocol procedure) may be created and tied to the protocol template (in this example, an Oxygen protocol template) created as described above. The protocol procedure may include any number of other procedures. For example, as described below, the example Oxygen protocol procedure includes both an ABG Procedure and an Oxygen procedure.

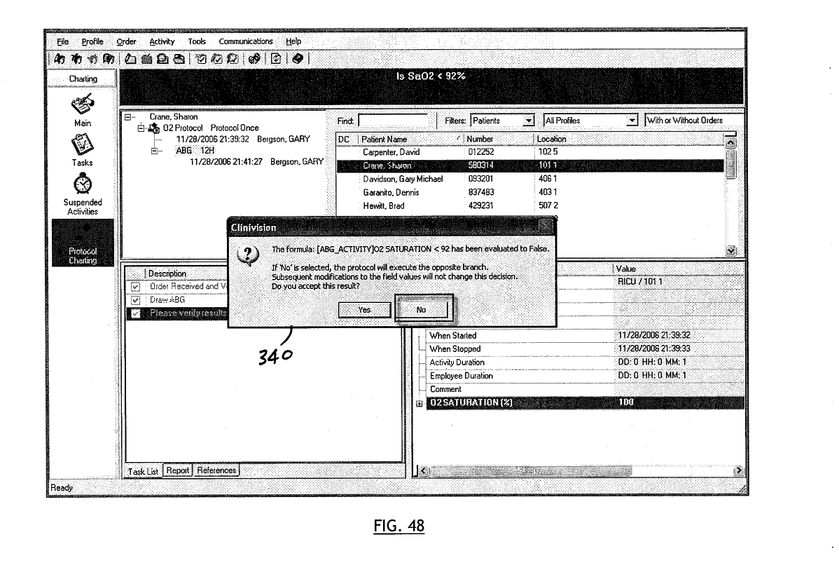

Before creating the protocol procedure, the user may identify template variables defined by the protocol template that need to be bound to objects (e.g., existing orders, order fields, activity fields, other fields, or other objects). Example template variables may include Order step names and variables in Decision step formulas. Binding a particular Order step name to a particular existing Order allows the system to automatically pull up the order form and the activity form corresponding to the particular existing Order when the particular Order step is encountered during run-time (i.e., charting of a patient using the protocol procedure), as discussed below regarding FIGS. 45-46 and 50-51. Binding a formula variable to a particular field (e.g., an order field, an activity field, or another field) allows the system to automatically pull the value in the particular field into the formula in order to process the Decision step formula when the Decision step is encountered during run-time, as discussed below regarding FIG. 48.

In this example, three template variables may be identified from the example Oxygen protocol template created above. The three template variables are shown below:

TABLE 1
Template variables to be bound

| | Variable | Object to which Template |
|---|---|---|
| Protocol Template Step | Name | Variable should be Bound |
| Step 2: ABG Order | abg | ABG Order |
| Step 3: Is SaO₂ < 92? | SaO₂ | Activity.O₂ SATURATION |
| Step 4: Oxygen Order | Oxygen | Oxygen Order |

FIGS. 24-25: Locating Fields in existing Procedures to be added to a Procedure Definition for a Protocol Procedure to be created. The user may locate fields in existing Procedures that will be added to the Protocol Procedure that will be created for the newly created Oxygen Protocol Procedure, as discussed below. Such fields may include, e.g., “O₂ Saturation,” “PH,” and “PEEP/CPAP.”

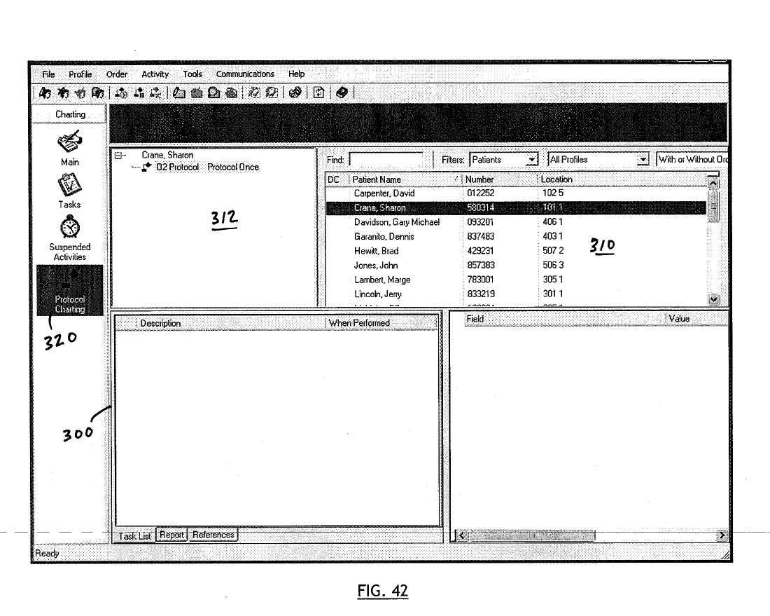

Suppose template variable “abg” (the user-defined name for the ABG Order step) should be bound to an ABG Order, as shown in Table 1. The user may be interested in a subset of fields associated with the ABG Order that may be relevant to the execution of the Oxygen Protocol (discussed below with reference to FIGS. 42-56). For example, the user may be interested in fields that may be relevant to Decision step formulas. To select the fields of interest, the user may open the ABG Procedure and locate and write down the field(s) of interest, as shown in FIGS. 24-25. In this example, one field of interest is the “O₂ Saturation” field, as the value of this field needs to be tied to the formula in Decision step 3.

As shown in FIG. 24, the user may select the “Procedure” icon 200 from the icons listed under the “Configuration” menu. This selection may open a listing a procedures maintained by software 44. The user may locate and select the existing ABG procedure. As shown in FIG. 25, this selection may open the procedure definition 202 for the ABG procedure. The procedure definition includes an order definition and an activity definition, as indicated by tabs 204 and 206, respectively. Because the user understands that relevant value for the variable SaO₂ in the Decision step 3 formula is the actual measured value, the user knows that the field of interest for the SaO₂ value will be located in the activity definition, rather than the order definition. (In other situations (e.g., for other formula variables), the field of interest may be located in the order definition or otherwise located.) Here, the user may select the activity definition and locate the field of interest: the “O₂ Saturation” field. The user may then record (e.g., write down) the name of the field: “O2 SATURATION.” As discussed below, the user may use this field name to bind the “O₂ Saturation” field of the existing ABG procedure to the Oxygen protocol procedure (which may be created as discussed below).

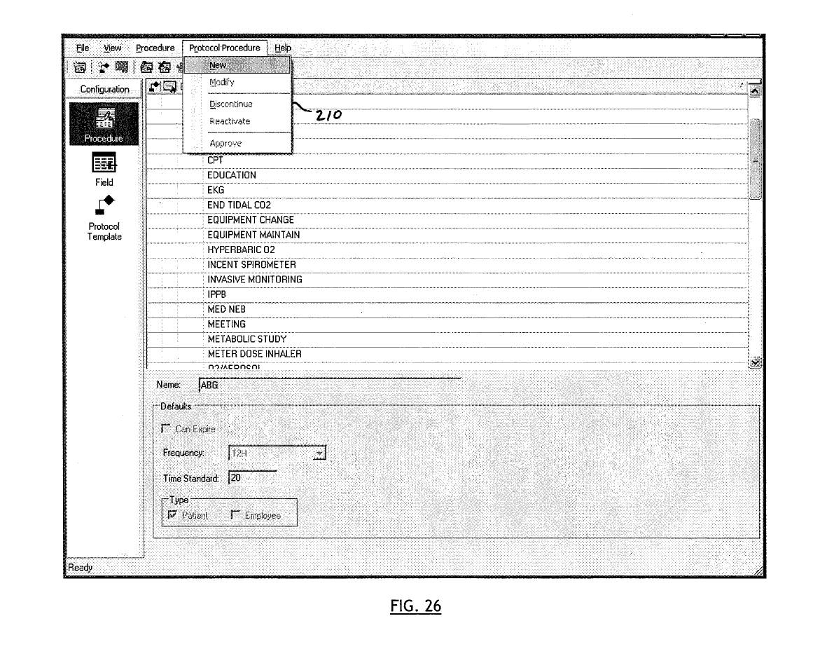

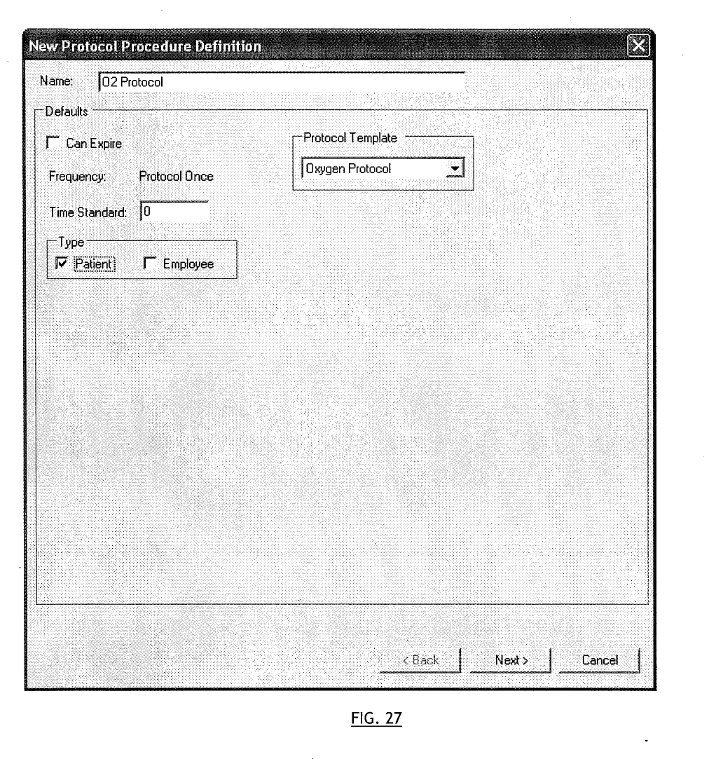

FIGS. 26-27: Creating a Protocol Procedure for Protocol Template. The user may now create a Protocol Procedure for the Oxygen protocol template created above. As shown in FIG. 26, the user may select “New” from a “Protocol Procedure” menu 210. This selection may bring up a “New Protocol Procedure Definition” window that allows the user to name the new protocol procedure and associate the new protocol procedure with a protocol template. As shown in FIG. 27, the user may name the new protocol procedure “O2 Protocol” and associate the new protocol procedure with the Oxygen Protocol template.

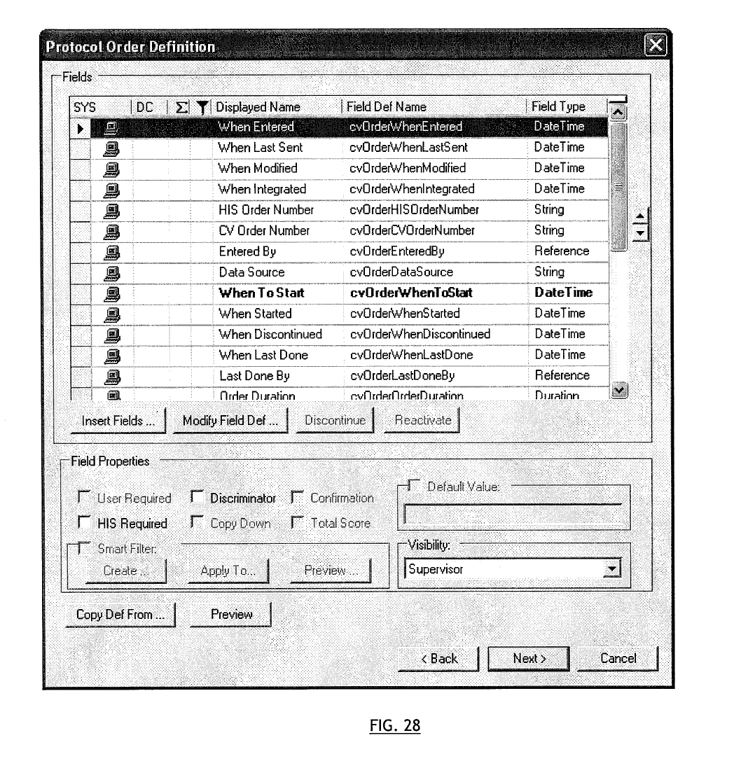

FIGS. 28-31: Adding Relevant Fields to the Protocol Procedure. The user may now add relevant fields to the protocol procedure for the newly created O2 Protocol Procedure. For example, the user may now add relevant fields to the protocol order definition and/or the protocol activity definition portions of the newly created O2 Protocol procedure. The new fields may include any fields that the user located in existing procedures as discussed above regarding FIGS. 24-25.

After naming the new protocol procedure definition (O2 Protocol) and associating it with the Oxygen protocol template (FIG. 27), the system may display the new protocol procedure definition, which may include (a) a protocol order definition and (b) a protocol activity definition. FIG. 28 illustrates an example protocol order definition form for the new O2 Protocol procedure. In this example, the user does not make any changes to the protocol order definition form. However, in other situations, the user may make any desired changes to the protocol order definition form.

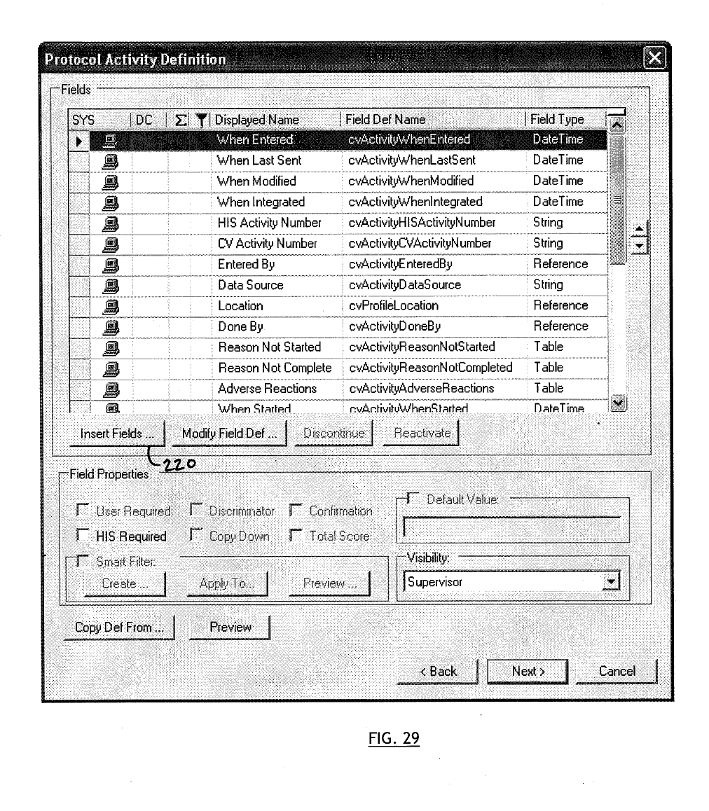

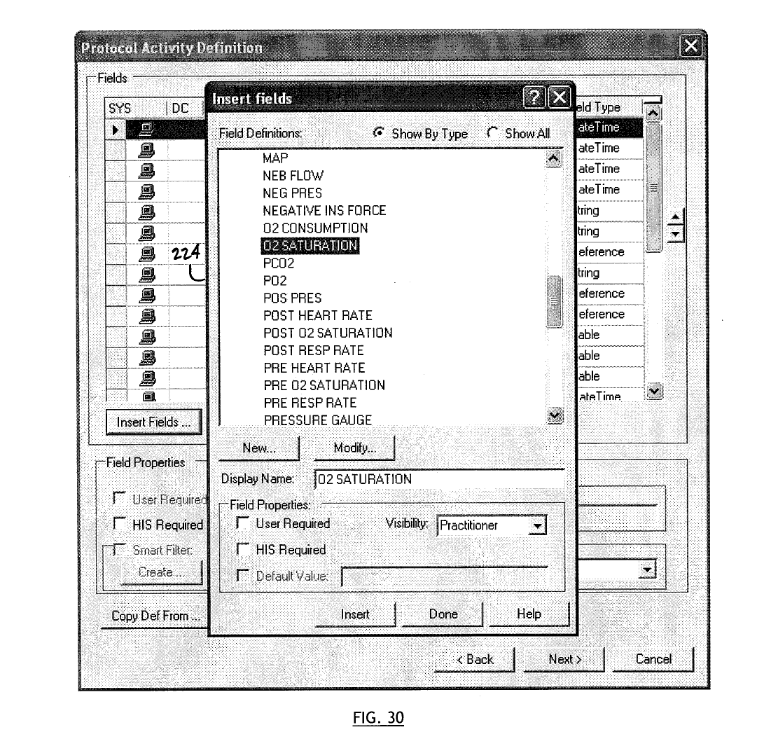

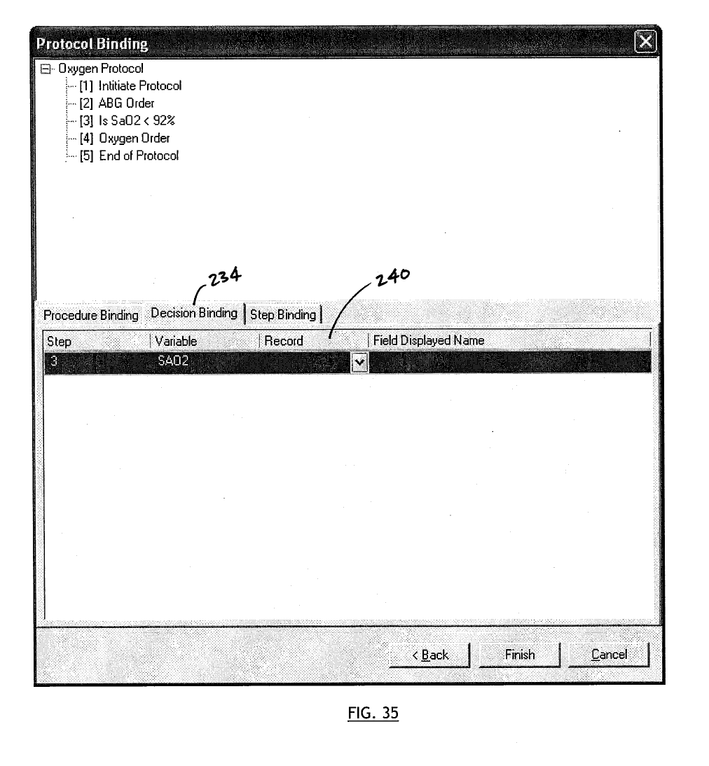

FIG. 29 illustrates an example protocol activity definition form for the new O2 Protocol procedure. In this example, the user wishes to add the “O₂ Saturation” field to the protocol activity definition such that the “O₂ Saturation” field may later be bound to the O2 Protocol procedure (as discussed below regarding FIGS. 35-37). To do this, the user may select the “Insert Fields” button 220, which may open an “Insert Fields” menu 224, as shown in FIG. 30. The “Insert Fields” menu 224 may display all (or some logical subset) fields included in any existing procedure definition, e.g., including any fields included in the existing ABG procedure definition, the existing Oxygen procedure definition, and any other existing procedure definition.

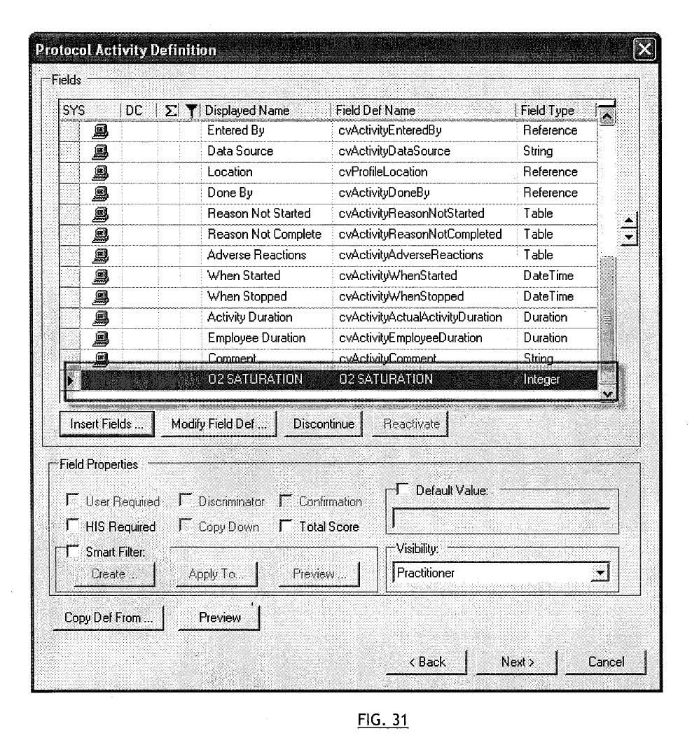

The user may then select the desired field—“O2 SATURATION”—and then select the “Insert” and “Done” buttons. As a result, the O2 SATURATION field is inserted into the protocol activity definition, as shown in FIG. 31. The user may repeat this process to add any desired fields into the protocol activity definition. Once the user is finished adding fields to the protocol activity definition, they may select the “Next” button to advance to a protocol binding form, in order to bind the protocol procedure.

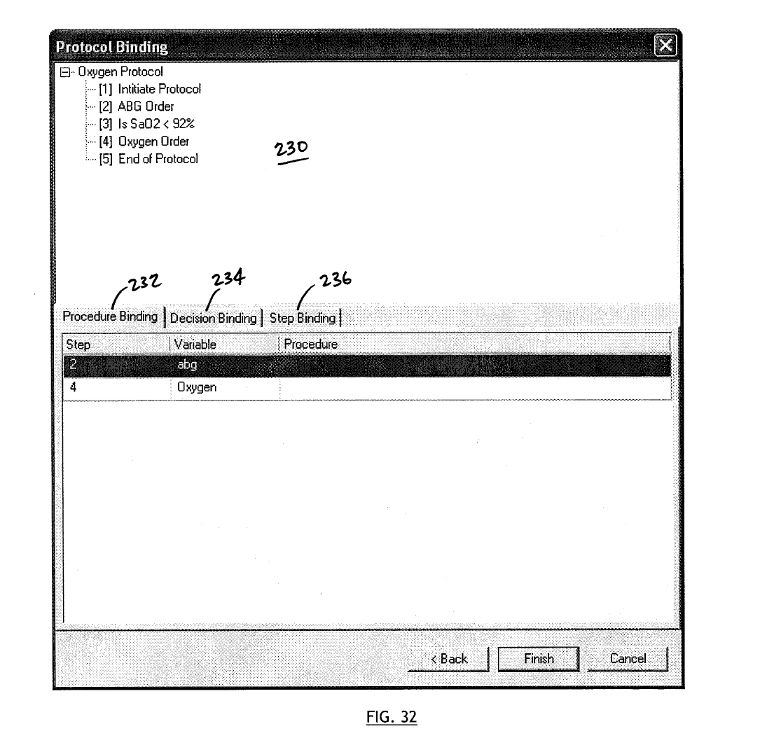

FIGS. 32-38: Binding the Protocol Procedure. FIG. 32 illustrates an example protocol binding form 230. In this example, binding form 230 indicates the five steps of the Oxygen Protocol created above, and includes three binding tabs: a Procedure Binding tab 232, a Decision Binding tab 234, and a Step Binding tab 236. These tabs may be used for binding the various aspects of the Oxygen Protocol to existing objects.

Procedure binding may be used to bind particular steps of the protocol—in particular, Order steps—to existing procedures. Thus, when an Order step is reached during run-time of the protocol, the system may automatically access the existing procedure bound to that Order step (e.g., to present to the user the Order form and/or Activity form associated with the accessed existing procedure).

Decision binding may be used to bind variables in Decision step formulas to existing fields. Thus, when a Decision step is reached during run-time of the protocol, the system may automatically access the value in each field that is bound to a formula variable, in order to process the formula.

Step binding may be used to bind one or more fields to a step of the protocol. During run-time of the protocol, when a step is encountered having one or more fields bound to that step via step binding, the system may prevent the user from moving beyond that step until data/values have been entered into all of the fields bound to that step. During run-time, if no data/value has been entered for one or more fields bound to the current step, the system may require the user (e.g., using prompts) to enter the missing data/values. For example, a user may with to use step binding to ensure that data/values have been entered for fields required for the execution of Decision steps.

As shown in FIG. 32, the Procedure Binding tab 232 is selected, which displays steps to be bound to existing procedures. Certain types of steps may be suitable for binding to existing procedures. In this example, Order steps (but not Start, End, or Decision steps) are suitable for binding to existing procedures, and thus Order steps 2 and 4 are displayed under Procedure Binding tab 232. The variables (abg and Oxygen) corresponding to the Order steps are also shown, which variables were previously entered in the “binding information: procedure name” fields during the building of the protocol template, as shown in FIGS. 12 and 16.

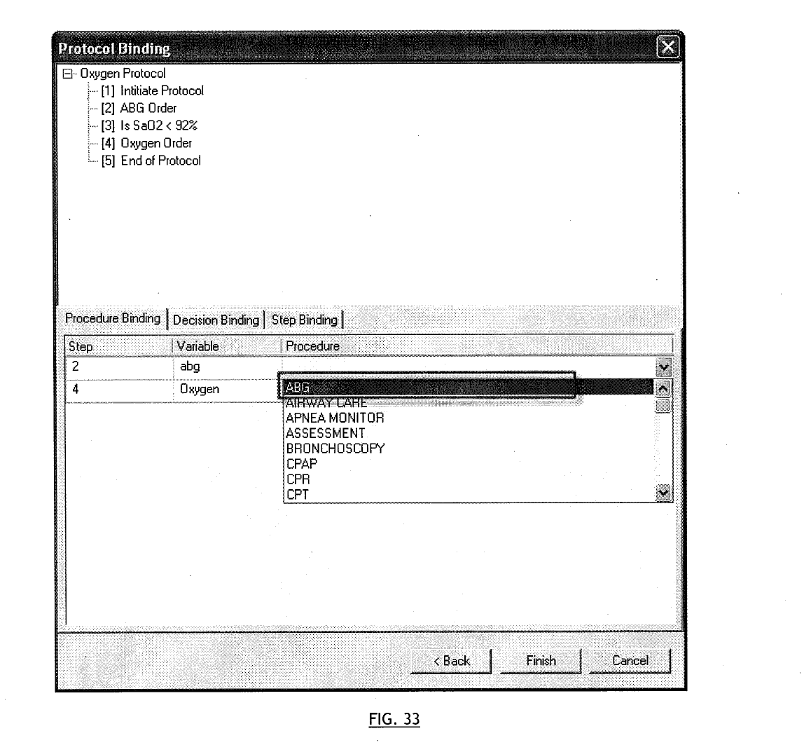

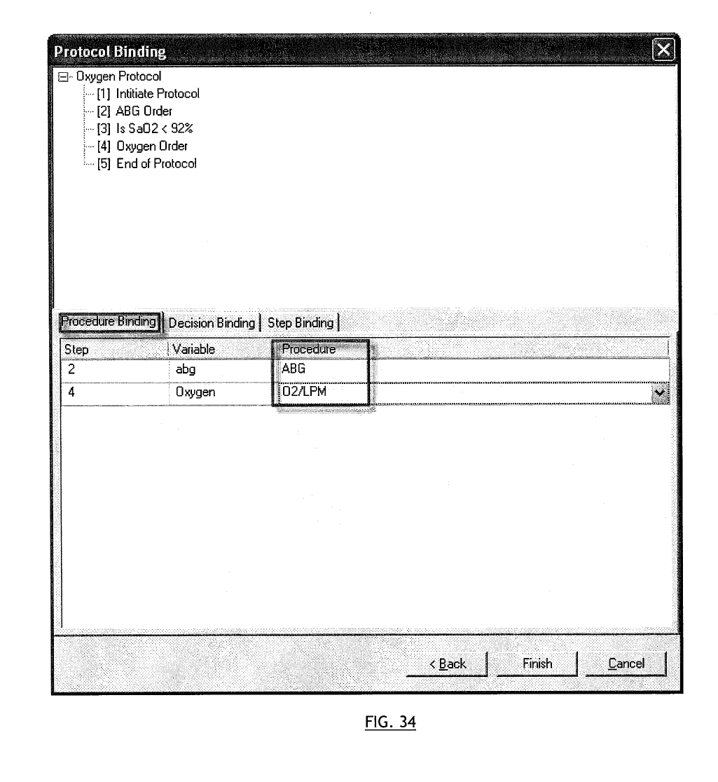

As shown in FIGS. 33 and 34, the user may select an existing procedure to bind to each of steps 2 and 4. In this example, the user binds the ABG procedure to step 2 (FIG. 33) and the O2/LPM procedure to step 4 (FIG. 34).

The user may then advance to decision binding by selecting Decision Binding tab 234, as shown in FIG. 35. Each variable from each Decision step in the protocol may be listed under Decision Binding tab 234, to be bound to an existing field. In this example, the Oxygen Protocol includes only a single Decision step (step 3) having a formula that includes only a single variable, SaO₂. Thus, “step 3” and variable “SAO2” are displayed under Decision Binding tab 234. The user wishes to bind the “SAO2” variable with the “O2 SATURATION” field located as discussed earlier with respect to FIGS. 24-25, such that during run-time, the software will import the value of the “O2 SATURATION” field into the formula “Is SaO₂<92?” to execute Decision step 3.

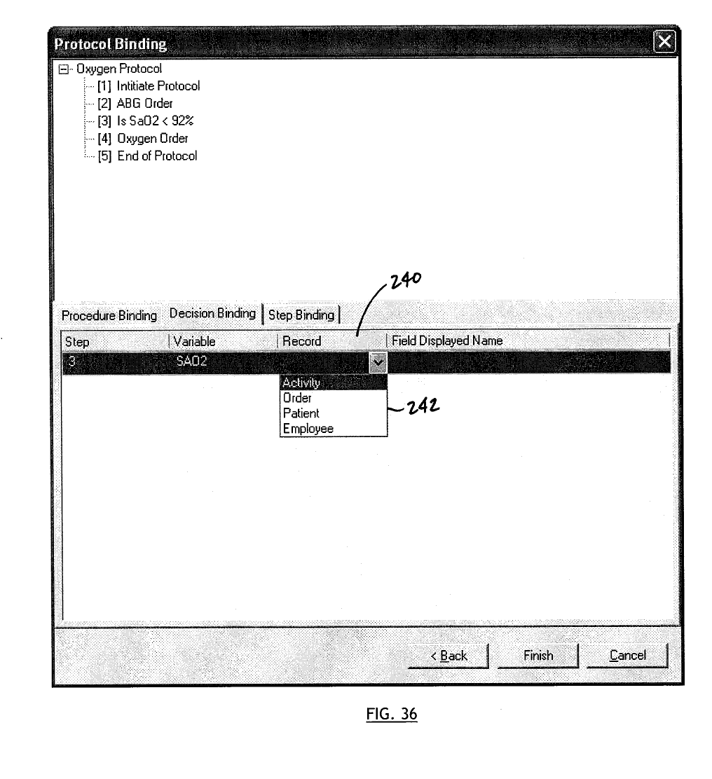

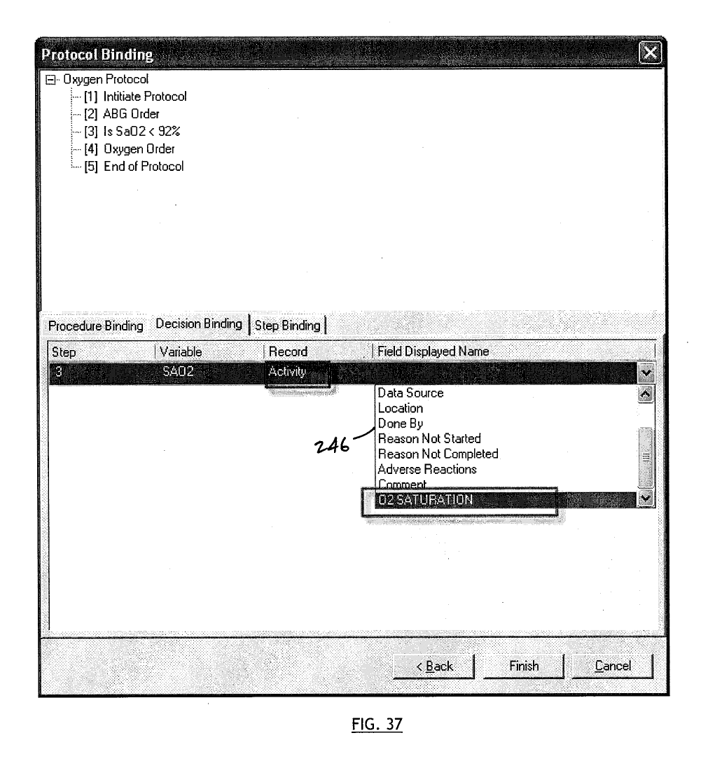

To select the field to bind to the SaO₂ variable, the user may first select the “Record” button 240, which opens a record menu 242 listing various sources of fields in which the desired field may be located, as shown in FIG. 36. Recalling that the user added the desired “O2 SATURATION” field to the protocol activity definition for the Oxygen protocol procedure (FIGS. 29-31), the user may select “Activity” from menu 242 to bring up a menu 246 of fields included in the protocol activity definition for the Oxygen protocol procedure, as shown in FIG. 37. The user may then locate and select the “O2 SATURATION” field, thus binding the “SAO2” variable with the “O2 SATURATION” field.

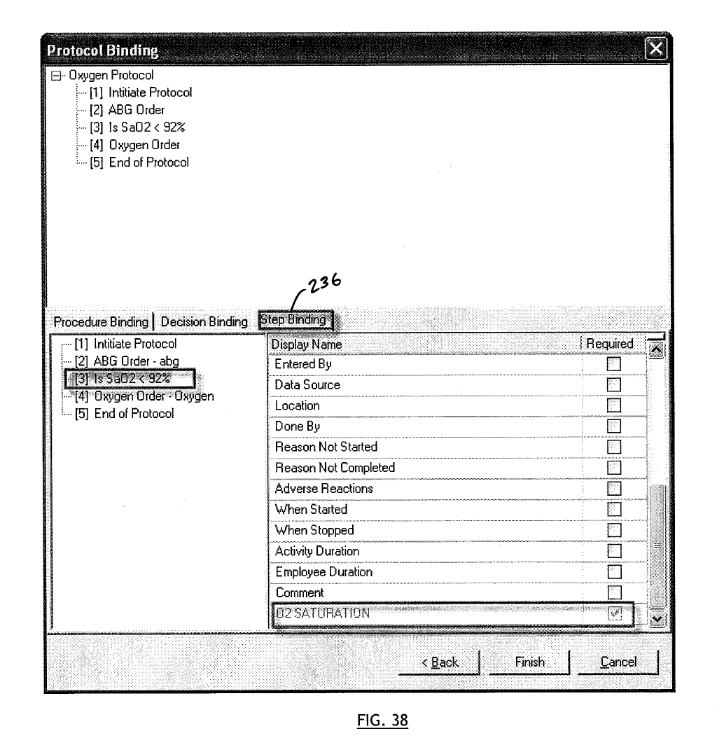

The user may then advance to step binding by selecting Step Binding tab 236, as shown in FIG. 38. Here, the user may bind one or more of steps 1-5 to one or more fields included in the protocol procedure definition (e.g., any fields included in the protocol order definition or protocol activity definition). To bind a particular step to a particular field, the user may select the particular step (here, step 3 is shown selected), and then the particular field to bind to that step.

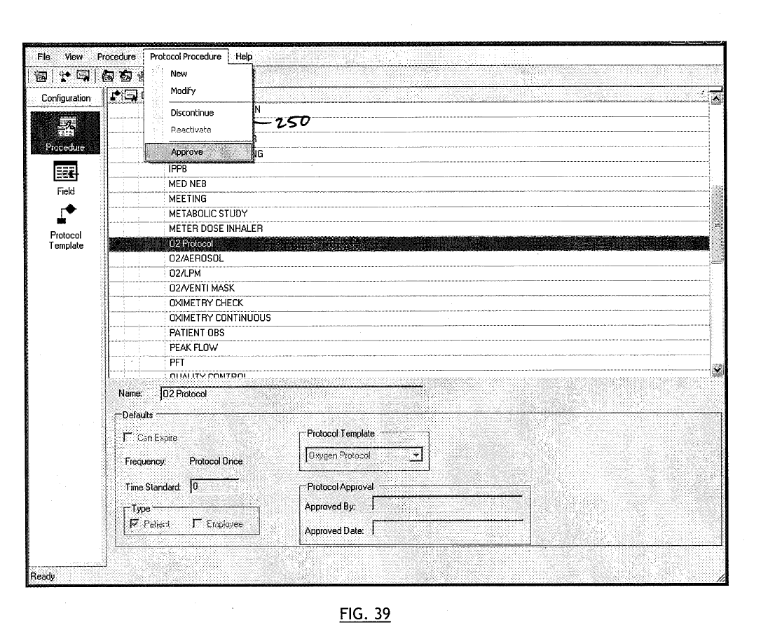

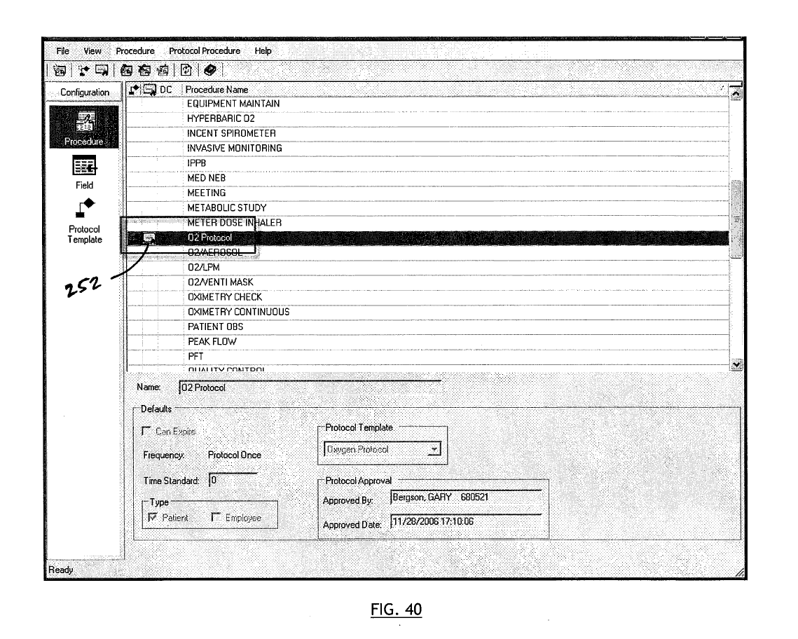

FIGS. 39-40: Approving the Protocol Procedure. As shown in FIG. 39, the user may approve the protocol procedure, e.g., by clicking “Approve” from Protocol Procedure menu 250. Once approved, the O2 Protocol procedure may be added to the list of existing procedures, as shown in FIG. 40. In some embodiments, an approval icon 252 is displayed to indicate that the protocol procedure has been approved.

The O2 Protocol is now ready for use for charting a patient, as discussed below.

Patient Charting Using the O2 Protocol Procedure

Once the protocol has been fully created, a user (e.g., therapist) may use the protocol to assist with the charting feature of the system/software. In some embodiments, the user may perform such charting at the patient's location (i.e., “bedside”) by using a mobile user device 30 having software 44 loaded thereon, as described above, for example.

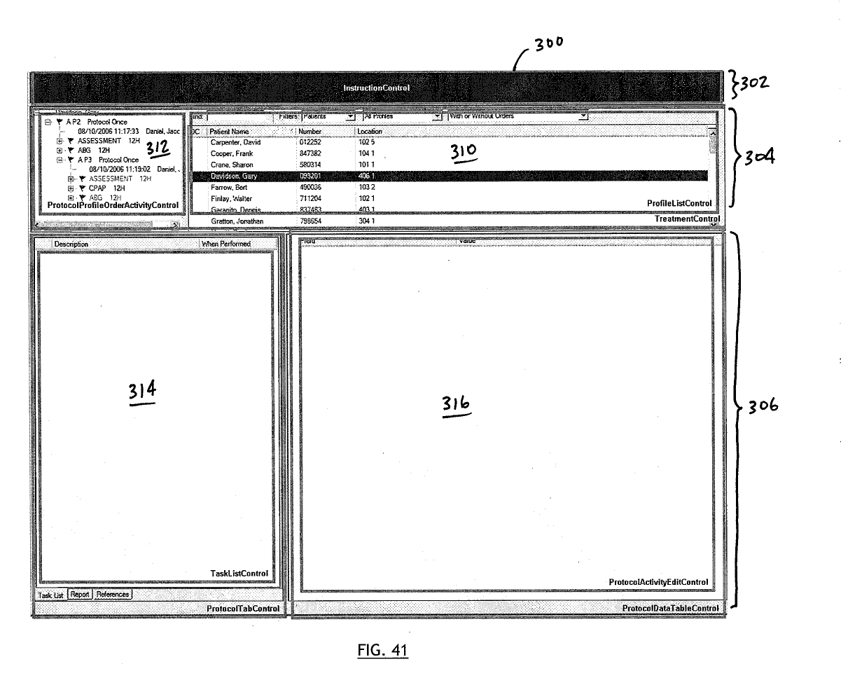

FIG. 41 illustrates an example display of a protocol charting workspace 300, according to one embodiment of the disclosure. In this example, protocol charting workspace 300 includes an instruction panel 302, a treatment panel 304, and a protocol property panel 306. Instruction panel 302 may be used to display user instructions (i.e., instructions to the user). Treatment panel 304 may include a profile list control panel 310 and a protocol profile order/activity control panel 312. Protocol property panel 306 may include a protocol tab control panel 314 and a protocol data table control panel 316.

As shown in FIG. 42, to begin charting a patient using a protocol, the user (e.g., a respiratory therapist) may select the “Protocol Charting” icon 320 from various icons listed under a “Charting” menu. This selection opens a protocol charting workspace 300. The user may select a patient for charting from the profile list control panel 310. Here, the user has selected the patient Sharon Crane. When the user selects a patient, one or more orders that have been entered for that patient are displayed in protocol profile order/activity control panel 312. Such orders may include individual orders and/or protocol orders (which may contain any number of individual orders). In this example, only a single order—the O2 Protocol order—has been entered for patient Sharon Crane.

Orders may be entered from various points in system 10 and received (e.g., downloaded) into the charting module shown in FIG. 42. For example, a doctor or other caregiver may enter order information for various patients via HIS 12. Such orders may then be communicated (e.g., downloaded) into the client software 44, as shown in FIG. 42. In this manner, orders may be automatically populated into panel 312 in connection with the selected patient.

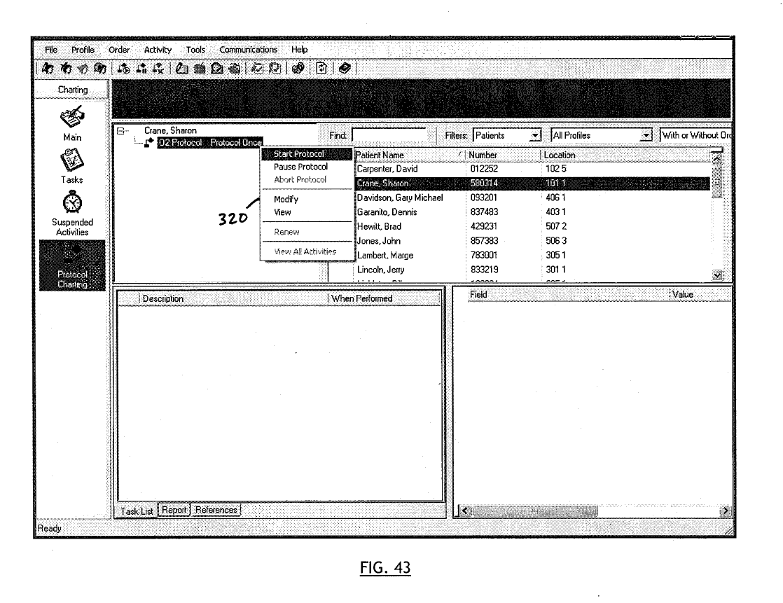

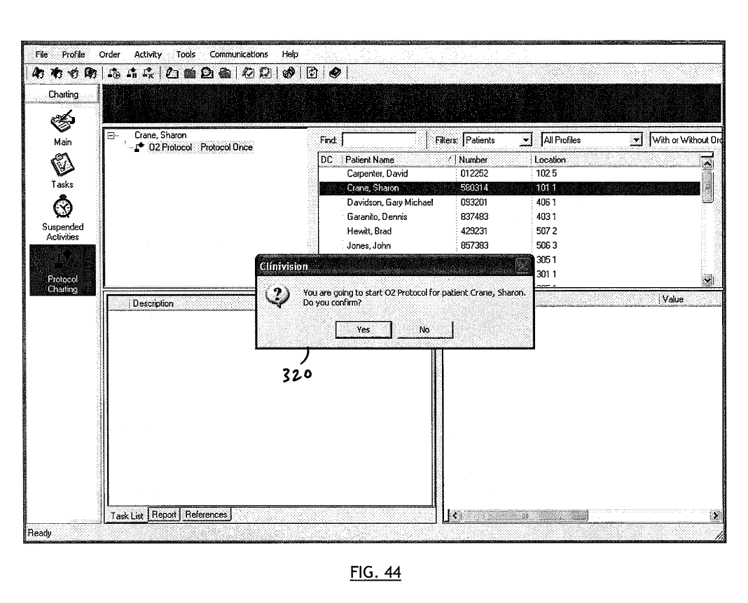

As shown in FIG. 43, in order to start the O2 Protocol, the user may click on the order, and then “Start Protocol” from a protocol action menu 320. As shown in FIG. 44, selecting “Start Protocol” may open a confirmation message 324 prompting the user to confirm the initiation of the O2 Protocol.

If the user confirms the initiation of the O2 Protocol, the protocol begins to execute the protocol template, beginning with “Step 1: Initiate Protocol” (see protocol template, FIG. 20). The protocol may then advance to “Step 2: ABG Order.” At this point, the system may access the existing ABG procedure (based on the binding created between Step 2 and the existing ABG procedure discussed above regarding FIGS. 32-33). As shown in FIG. 44, the system may then display an ABG order form 194 and prompt the user to create an ABG order, e.g., by filling in one or more fields in order form 194. Once the user has filled in the fields as desired, she may click “Save.”

As shown in FIG. 46, the system may then display an ABG activity form 197 and prompt the user to create an ABG activity, e.g., by filling in one or more fields in activity form 197. In order to obtain data to enter into the various fields of form 197, the user may perform one or more tests, take measurements, make observations of the patient, read data/values from a ventilator screen or other medical device, etc. In some embodiments, at least a portion of such data may be automatically communicated from the ventilator or other medical device(s) into form 197 (e.g., via wireless or wireline links between the ventilator/medical device and user device 30). In this example, the user may read 100% O2 saturation from the ventilator screen or from an oximeter display, and enter that value into the “O2 SATURATION” field. Once the user has filled in the fields as desired, she may click “Save.”

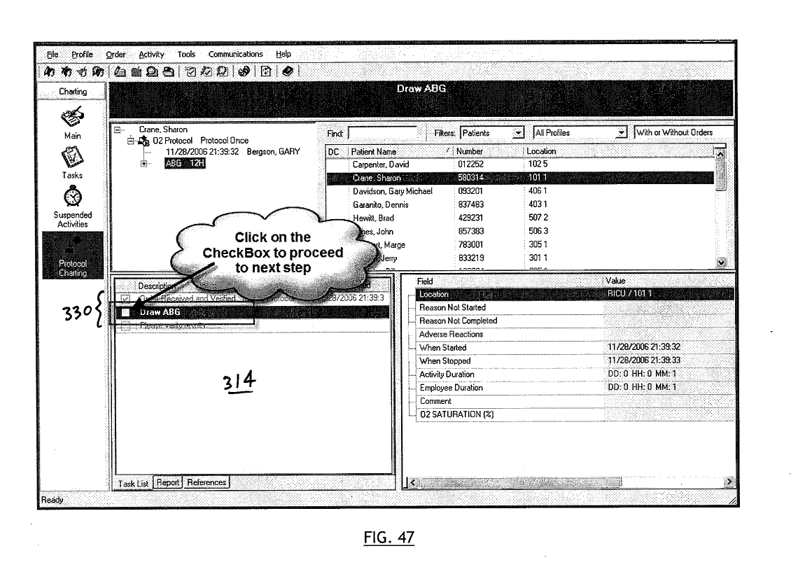

As shown in FIG. 47, a task list 330 may be displayed in protocol tab control panel 314. Task list 330 may include a number of task list statements associated with the execution of the protocol. Such task list statements may track the protocol steps as the user advances through the process flow of the protocol. As the user processes or finishes each step, software 44 may display one or more task list statements for that step. The task list statements displayed in task lists 330 are the task list statements defined/entered by the user for each step during the building of the protocol template (e.g., see FIGS. 10, 12, 14, 16, and 18).

The user may check off each task list statement as each step/task is completed. In some embodiments, certain task list statements may be automatically checked (e.g., the task list statement associated with a Start step). When the user checks off the task list statement associated with a particular step, the protocol may automatically advance to the next step. For example, in the example shown in FIG. 47, the user has just completed “Step 2: ABG Order,” so the task list statement entered by the user for Step 2 (see FIG. 12), namely “Draw ABG,” is displayed in task list 330 with a blank box. The user may then check the box (e.g., by clicking on the box) to advance to Step 3.

After the user checks the box for “Draw ABG” in task list 330, the protocol advances to “Decision Step 3: Is SaO₂<92?” Software 44 automatically retrieves values for any variable in the formula, based on the decision binding discussed above with reference to FIGS. 35-37. Software 44 may then determine the True/False result of the decision. In this example, software retrieves the value 100 from the “O2 SATURATION” field linked to the SaO₂ variable in the formula, and determines the decision to be False.

Software 44 may then check the setting for “User Confirmation Required” that is associated with Decision step 3 (which setting may be user-selectable, as discussed above regarding FIG. 13). If the setting is False, the protocol may automatically advance to the next step according to the result of the decision (in this case, the decision is False, so the protocol may automatically advance along the “No” path of the protocol process flow, i.e., to “Step 5: End of Protocol”).

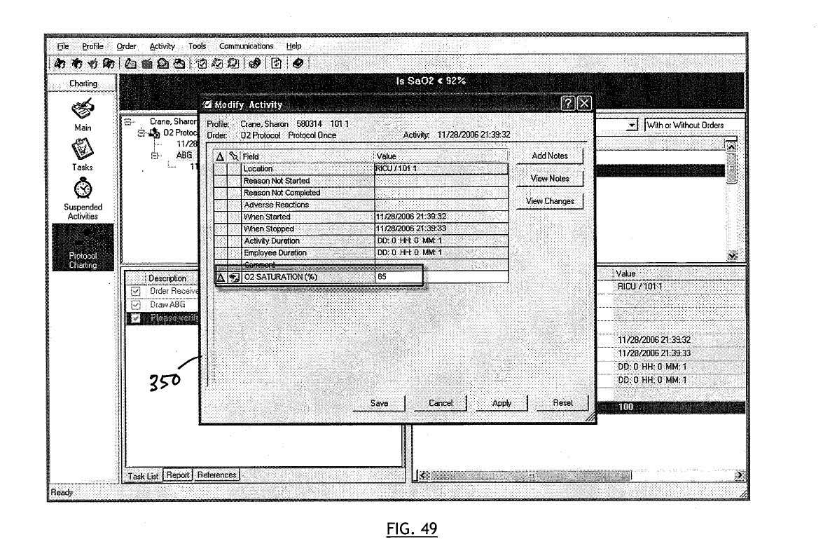

However, if the setting for “User Confirmation Required” associated with Decision step 3 is True (which is the case in this example, as shown in FIG. 13), software 44 may prompt the user to confirm the result of Decision step 3. For example, as shown in FIG. 48, a confirmation prompt 340 may be displayed that asks the user whether to accept the result (False) of the decision. If the user clicks “Yes” to accept the decision, the protocol will advance to the next step according to the result of the decision (in this case, to “Step 5: End of Protocol”). However, if the user clicks “No” to not accept the decision, the protocol may advance to the opposite branch (in this case, along the “Yes” path to “Step 4: Oxygen Order”), which is shown in FIG. 50. In some embodiments, before advancing to the opposite branch, software 44 may allow the user to modify or override the values for one or more activity fields. For example, as shown in FIG. 49, the system may display a modify activity form 350 (which may be similar to activity form 197 shown in FIG. 46) to allow the user to modify or override one or more field values. In this example, the user recognized that the 100% value for O2 SATURATION was erroneous, and the correct value is 65%. Thus, the user edits the value to 65% in form 350. It should be understood that in this embodiment, the edited values are relevant for record-keeping purposes, but do not affect the decision made at Step 3.

In this manner, the user may be given the option to override automatic decisions made at Decision steps. As discussed above, this feature may be useful, for example, in situations where the user has additional knowledge about the patient, treatment, erroneous entered data (as in the example discussed above), etc. that may not be factored into the automated decision or that may result in an automated decision that the user believes to be inappropriate.

In response to a True result at Decision step 3, or as a result of the user overriding a False result at Decision step 3 (as discussed above), the protocol may then advance to “Step 4: Oxygen Order,” as shown in FIG. 50. At this point, the system may access the existing Oxygen procedure (based on the binding created between Step 4 and the existing Oxygen procedure discussed above regarding FIGS. 32-34). As shown in FIG. 50, the system may then display an Oxygen order form 196 and prompt the user to create an Oxygen order, e.g., by filling in one or more fields in order form 196. Once the user has filled in the fields as desired, she may click “Save.”

As shown in FIG. 51, the system may then display an Oxygen activity form 198 and prompt the user to create an Oxygen activity, e.g., by filling in one or more fields in activity form 198. In order to obtain data to enter into the various fields of form 198, the user may perform one or more tests, take measurements, make observations of the patient, read data/values from a ventilator screen or other medical device, etc. In some embodiments, at least a portion of such data may be automatically communicated from the ventilator or other medical device(s) into form 198 (e.g., via wireless or wireline links between the ventilator/medical device and user device 30). In the example shown in FIG. 51, the user may select “CANNULA” for the O2 DEVICE/LPM field after connecting a cannula to the patient. Once the user has filled in the fields as desired, she may click “Save.”

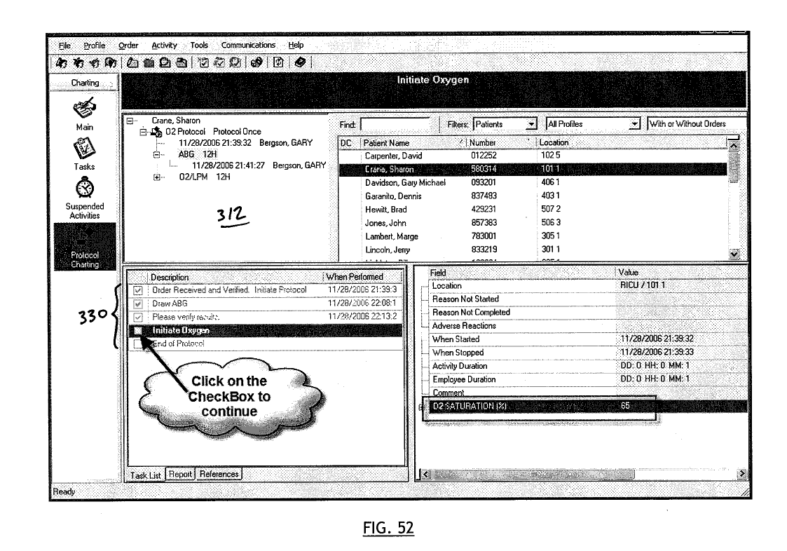

As shown in FIG. 52, when the user completes “Step 4: Oxygen Order,” the task list statement entered by the user for Step 4 (see FIG. 16), namely “Initiate Oxygen,” is displayed in task list 330 with a blank box. The user may then check the box (e.g., by clicking on the box) to advance to Step 5. It is also noted that as the various steps are completed, including the completion of various orders, the records of such orders being completed is recorded in protocol profile order/activity control panel 312.

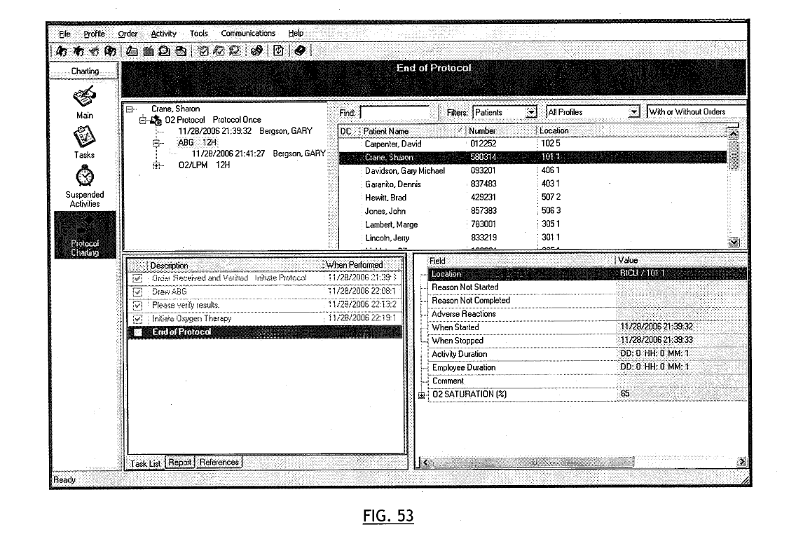

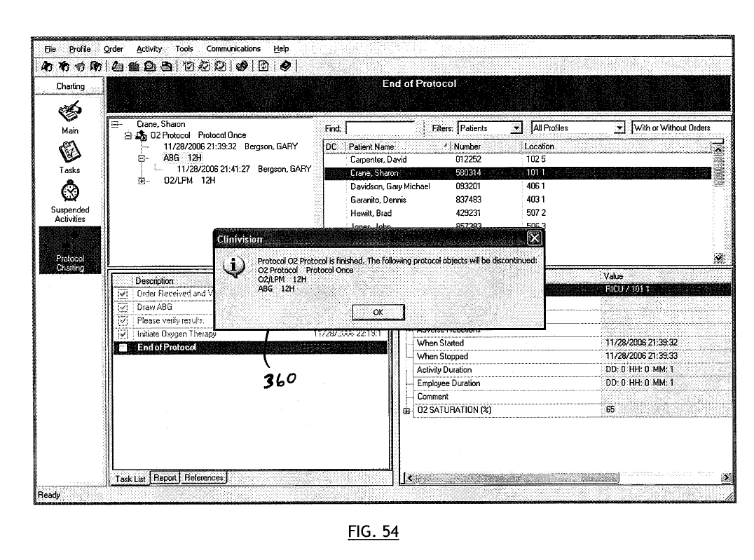

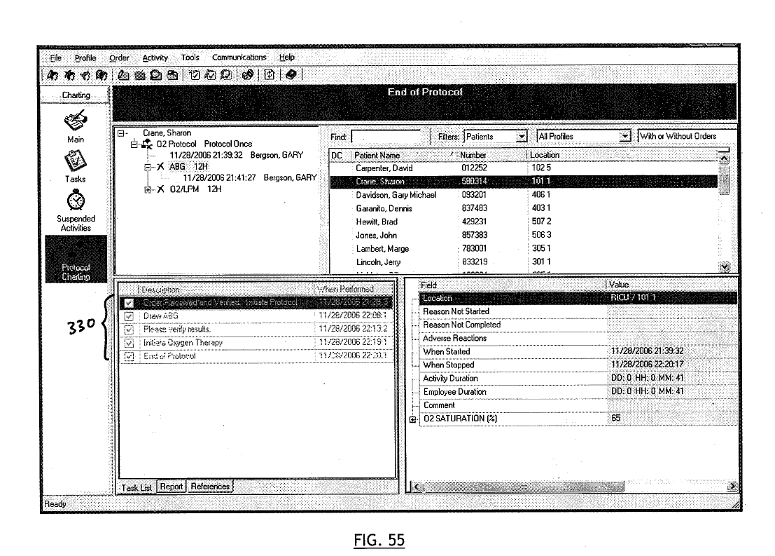

After the user checks the box for “Initiate Oxygen” in task list 330, the protocol advances to “Step 5: End of Protocol.” As shown in FIG. 53, the “End of Protocol” task statement may then be displayed in task list 330 with a blank box. The user may then check the box (e.g., by clicking on the box), followed by clicking in a confirmation window 360 the end of the protocol, as shown in FIG. 54. FIG. 55 shows the resulting final screen, with all items in task list 330 checked and grayed.

Protocol Reporting

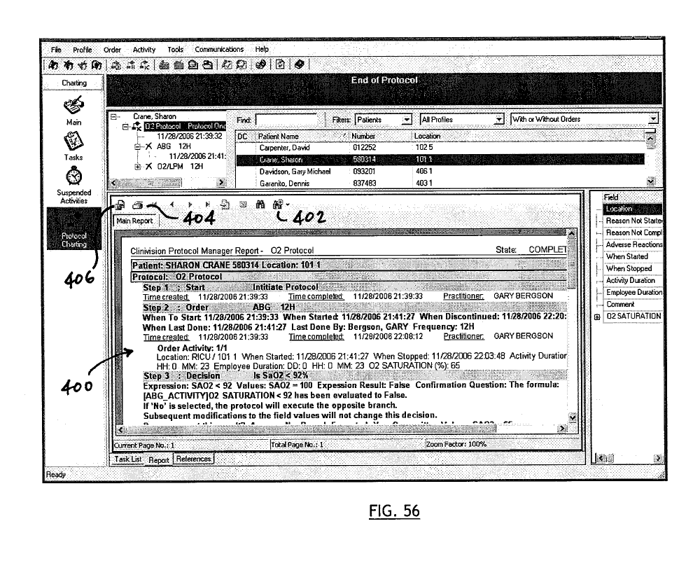

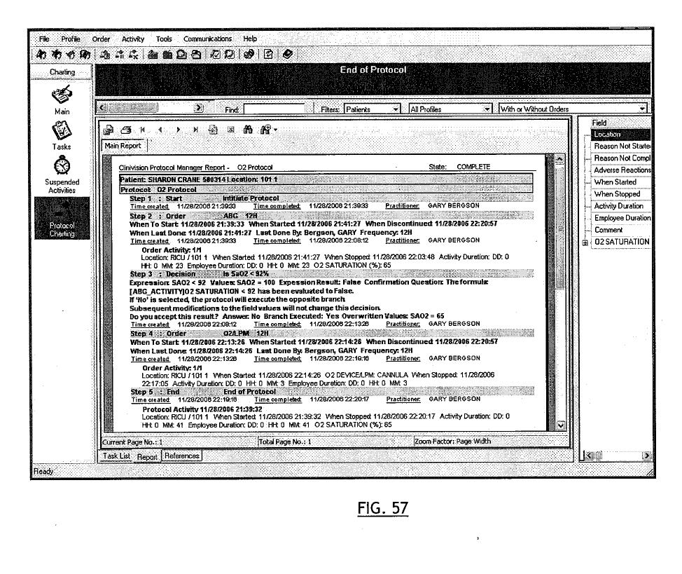

In some embodiments, software 44 may also provide reporting functions in order to generate reports regarding a protocol executed for a patient. FIGS. 56 and 57 illustrate screenshots of example reports, according to one embodiment of the disclosure. FIG. 56 illustrates a report 400 for the O2 Protocol completed for patient Sharon Crane, e.g., as discussed above. A zoom icon 402 may be selected by the user to zoom in on the report to a desired magnification or to provide a full page view of the report, e.g., as shown in FIG. 57. A print icon 404 may be selected by the user to print a copy of the report. An export icon 406 may be selected by the user to export a report file or files to another software application or computing device.

FIGS. 58-61 illustrate additional example medical protocols that may be configured and implemented using any of the techniques discussed above.

Although the disclosed embodiments have been described in detail, it should be understood that various changes, substitutions and alterations can be made herein without departing from the spirit and scope of the disclosure as illustrated by the following claims. For example, it should be understood that in various embodiments, system 10 may include any combination of one, some or all of the various components and/or features discussed above and/or any one or more additional components and/or features. As another example, it should be understood that the methods discussed above are examples only, and that methods according to the present disclosure may include any combination of some or all of the steps discussed above, including any suitable variations of such steps, and may be performed in any suitable order. In addition, multiple steps may be performed partially or completely simultaneously.

What is claimed is:

1. A method for configuring a protocol for use in providing a medical treatment to a patient, the method comprising:
selecting a protocol to be configured, the protocol being associated with a medical treatment having a process flow including multiple steps;
creating a protocol template for the protocol, including:
selecting and arranging flowchart components to create a graphical flowchart representation of the process flow; and
defining one or more variables for one or more steps in the process flow; and
linking the one or more variables to one or more existing objects such that when the protocol is executed, the existing objects are automatically accessed to facilitate execution of the protocol.

2. A method according to claim 1, wherein linking the one or more variables to one or more existing objects comprises linking a particular variable associated with a particular step of the process flow to one or more existing data fields such that data in the one or more existing data fields is automatically accessed when the particular step is executed.

3. A method according to claim 1, wherein:
the process flow includes a decision step having an associated formula including one or more formula variables; and
linking the one or more variables to one or more existing objects comprises linking the one or more formula variables associated with the decision step to one or more existing data fields such that when the formula step is executed, data in the one or more existing data fields is automatically accessed and used for the one or more formula variables.

4. A method according to claim 1, wherein:
the process flow includes an order step;
defining one or more variables for one or more steps in the process flow comprises defining a variable name for the order step; and
linking the one or more variables to one or more existing objects comprises linking the variable name for the order step to an existing order such that when the order step is executed, data regarding the existing order is automatically accessed and displayed.

5. A method according to claim 1, wherein the protocol is associated with respiratory therapy.

6. A method according to claim 1, wherein selecting and arranging flowchart components to create a graphical flowchart representation of the process flow comprises selecting and arranging flowchart components from a library of predefined types of flowchart components.

7. A method according to claim 1, wherein:
the process flow includes a decision step;
the method further comprises selecting a setting for a user confirmation feature, the user confirmation feature operable, during execution of the protocol, to prompt a user for confirmation of an automatic decision made at the decision step before advancing to a subsequent step in the process flow.

8. A system for configuring a protocol for use in providing a medical treatment to a patient, the system including software encoded in computer-readable media and when executed by a processor, operable to:
provide a user interface for creating a protocol, the protocol associated with a medical treatment having a process flow including multiple steps;
provide a user interface for creating a protocol template for the protocol, including:
a user interface for selecting and arranging flowchart components to create a graphical flowchart representation of the process flow; and
a user interface for defining one or more variables for one or more steps in the process flow; and
provide a user interface for linking the one or more variables to one or more existing objects such that when the protocol is executed, the existing objects are automatically accessed to facilitate execution of the protocol.

9. A system according to claim 8, wherein providing a user interface for linking the one or more variables to one or more existing objects comprises providing a user interface for linking a particular variable associated with a particular step of the process flow to one or more existing data fields such that data in the one or more existing data fields is automatically accessed when the particular step is executed.

10. A system according to claim 8, wherein:
the process flow includes a decision step having an associated formula including one or more formula variables; and
providing a user interface for linking the one or more variables to one or more existing objects comprises providing a user interface for linking the one or more formula variables associated with the decision step to one or more existing data fields such that when the formula step is executed, data in the one or more existing data fields is automatically accessed and used for the one or more formula variables.

11. A system according to claim 8, wherein:
the process flow includes an order step;
the user interface for defining one or more variables for one or more steps in the process flow comprises a user interface for defining a variable name for the order step; and
providing a user interface linking the one or more variables to one or more existing objects comprises linking the variable name for the order step to an existing order such that when the order step is executed, data regarding the existing order is automatically accessed and displayed.

12. A system according to claim 8, wherein the protocol is associated with respiratory therapy.

13. A system according to claim 8, wherein the user interface for selecting and arranging flowchart components to create a graphical flowchart representation of the process flow comprises a user interface for selecting and arranging flowchart components from a library of predefined types of flowchart components.

14. A system according to claim 8, wherein:
the process flow includes a decision step;
the method further comprises providing a user interface for selecting a setting for a user confirmation feature, the user confirmation feature operable, during execution of the protocol, to prompt a user for confirmation of an automatic decision made at the decision step before advancing to a subsequent step in the process flow.

15. A method for using a protocol for facilitating a medical treatment for a patient, the method comprising:
initiating execution of a protocol, the protocol associated with a medical treatment and having a process flow including multiple steps, wherein a particular step has one or more associated variables linked to one or more existing objects;
executing the steps of the process flow; and
during execution of the particular step, automatically accessing the existing objects linked to the one or more variables associated with the particular step in order to facilitate execution of the particular step.

16. A method according to claim 15, wherein:
the particular step comprises a decision step having an associated formula including one or more formula variables linked to one or more existing data fields; and
during execution of the decision step, automatically accessing the existing data fields linked to the one or more formula variables in order to process the formula.

17. A method according to claim 15, wherein:
the particular step comprises an order step having a variable name linked to an existing order; and
during execution of the order step, automatically accessing the existing order linked to the variable name for the order step and displaying data regarding the accessed existing order.

18. A method according to claim 15, wherein the protocol is associated with respiratory therapy.

19. A method according to claim 15, wherein:
the particular step comprises a decision step; and
the method further comprises, after an automatic decision associated with the decision step has been made, prompting a user for confirmation of the automatic decision before advancing to a subsequent step in the process flow.

20. A system for configuring a protocol for use in providing a medical treatment to a patient, the system comprising:
means for creating a protocol, the protocol associated with a medical treatment having a process flow including multiple steps;
means for creating a protocol template for the protocol, including:
means for selecting and arranging flowchart components to create a graphical flowchart representation of the process flow; and
means for defining one or more variables for one or more steps in the process flow; and
means for linking the one or more variables to one or more existing objects such that when the protocol is executed, the existing objects are automatically accessed to facilitate execution of the protocol.
